(19) United States
(12) Patent Application Publication
(10) Pub. No.: US 2024/0094221 A1
(43) Pub. Date: Mar. 21, 2024

(54) METHOD FOR PREDICTING THE FERTILITY POTENTIAL OF A FEMALE SUBJECT

(71) Applicant: Rigshospitalet, Copenhagen Ø (DK)

(72) Inventor: Martin Blomberg Jensen, Charlottenlund (DK)

(21) Appl. No.: 18/265,584

(22) PCT Filed: Dec. 8, 2021
(86) PCT No.: PCT/DK2021/050358

§ 371 Date: Jun. 6, 2023

(30) Foreign Application Priority Data
Dec. 8, 2020 (DK) PA 2020 70817

(51) Int. Cl. G01N 33/68; A61K 31/593; A61K 38/24; A61K 38/29; A61P 15/08

(52) U.S. Cl. CPC G01N 33/689; A61K 31/593; A61K 38/24; A61K 38/29; A61P 15/08; G01N 2333/70575; G01N 2333/70578; G01N 2800/367; G01N 2800/52

(57) ABSTRACT

The present invention relates to methods for predicting the fertility potential of a female subject using RANKL and OPG as biomarkers. The present invention also relates to the use of stimulators of RANKL, inhibitors of RANKL and/or inhibitors of OPG to treat female infertility.

### TECHNICAL FIELD OF THE INVENTION

The present invention relates to methods for predicting the fertility potential of a female subject using RANKL and OPG as biomarkers. The present invention also relates to the use of stimulators or inhibitors of RANKL or inhibitors of OPG to treat female infertility.

### BACKGROUND OF THE INVENTION

Infertility is a prevalent condition and the cause may be female, male, a combination, or unexplained. 12 months of unprotected intercourse without contraception and without conception is defined as infertility. Infertility has plagued humans throughout history, but today advanced assisted reproductive technology has aided the diagnosis of infertility and revolutionized the treatment. In women, several sites are important for optimal female fertility. A hostile endometrial environment will impede embryonic implantation as shown by the placement of an intrauterine device into the endometrial cavity for contraception. Other factors, which have been implicated in the development of a hostile environment for implantation, are autoimmune factors such as lupus anticoagulant and Integrin IIIβ. In addition to the endometrium and cervix, the shape and competency of the uterine fundus for instance due to anomalies of mullerian fusion and fibroids may impair implantation or growth of the pregnancy. Clearly, the tube must be open to allow fertility and the tube is also important to sweep the oocyte into the tube where it is fertilized by the sperm. Most importantly, pregnancy is direct evidence of ovulation. Patients who do not ovulate cannot conceive without assisted reproductive technology. Ovulatory function requires the integration of many normally functioning systems. Normal thyroid function, normal insulin action, normal adrenal function and perhaps normal cerebral function are all required for ovulation.

AMH or anti-mullerian hormone is used to assess a woman's ovarian reserve or egg count. AMH is a hormone produced by cells from the small follicles in a woman's ovaries and is used as a marker of oocyte quantity.

RANK/RANKL triggers a network of TRAF-mediated kinase cascades that promote osteoclast differentiation. RANKL is expressed on osteoblast cells and its receptor, RANK, on pre-osteoclastic cells. RANKL expression is stimulated by a number of factors, such as IL-1, IL-6, IL-11, IL-17, TNF-α, vitamin D, Ca²⁺, parathyroid, glucocorticoids, prostaglandin E2, and immunosuppressive drugs, and is down-regulated by TGF-α. The RANK/RANKL interaction induces differentiation and formation of multinucleated mature osteoclasts, causing bone resorption. The third protein, osteoprotegerin (OPG), is produced by fibroblasts in the skeleton and is known to exert an inhibitory effect on the pre-osteoclastic differentiation process. By binding to RANKL also known as osteoprotegerin binding protein (OPGbp), OPG inhibits the RANK/RANKL interaction and subsequent osteoclastogenesis. OPG is thus a very efficient anti-resorptive agent. It also serves as a decoy receptor for the tumour necrosis factor-related apoptosis-inducing ligand (TRAIL) and increases cell survival by blocking the apoptotic effects of this ligand and RANKL. The fact that the overexpression of OPG in mice results in severe osteopetrosis and that OPG-null mice are osteoporotic is a testimony to the physiological importance of OPG. Likewise, the lack of RANK or RANKL induces osteopetrosis in mice.

EP 3 030 249 B1 discloses a method for treating male infertility using a RANKL inhibitor such as Denosumab or OPG. EP 3 030 249 B1 is silent in respect of treating female infertility and is also silent in respect of using an activator of RANKL to treat female infertility.

EP 3 244 911 B1 discloses a method for determining a likely effect of a treatment to improve male fertility. EP 3 244 911 B1 is silent in respect of treating female infertility and is also silent in respect of using an activator of RANKL to treat female infertility.

EP 2 567 236 B1 relates to a method for predicting the fertility potential in a male mammal using OPG levels in a blood serum sample as a biomarker. EP 2 567 236 B1 is silent in respect of treating female infertility and is also silent in respect of using an activator of RANKL to treat female infertility.

Hence, an improved method for predicting female fertility potential would be advantageous, and in particular a more efficient and/or reliable method to treat female infertility or improve female fertility potential would be advantageous.

### Summary of the Invention

Here our data show that the RANKL signaling system is present in the female reproductive organs and important for female reproductive function by exerting effects on the number of follicles, maturation of follicles and uterus function. Measuring RANKL in biological fluids can help guide assisted reproductive techniques such as IVF and ICSI, select the oocytes that should be transferred back after fertilization in vitro. Moreover, manipulation of the RANKL signaling system in the ovary and uterus by using RANKL stimulators or inhibitors or OPG inhibitors may be used to improve female reproductive function.

The RANKL system is different in the female compared with the male gonads. In the testis there is high expression of both RANKL and OPG. In the ovary there is high OPG expression and low RANKL expression. This indicates that OPG is the main determinant for RANKL activity in the ovary while both OPG and RANKL changes may lead to altered activity in testis. Therefore, the task in the ovary in most clinical situations is to reduce OPG or stimulate RANKL, while in the reproductive tract in both sexes there is a similar expression and here RANKL inhibition seems to be beneficial. Therefore, the use of a RANKL stimulator or RANKL inhibitor is more dependent on the disease in the reproductive system.

Thus, the present invention is based on the realization that the RANKL/OPG/RANK system is present in the female reproductive system, and that RANKL and OPG levels may be indicative of the female fertility potential (see example 1 (mice data) and examples 2-3 (human data). Interestingly, the system appears to work oppositely regulated compared for what has previously been reported for the male reproductive system, in the sense that blocking RANKL-RANK interactions in males appear to improve the male fertility potential whereas, as reported here, promoting RANKL-RANK interactions appear to promote the female fertility potential. However, in women with specific diseases such as abnormal maturation of many follicles such as polycystic ovarian syndrome or repeated abortions due to impaired implantation in uterus short term RANKL inhibition may increase the chance for a pregnancy and live birth.

Thus, an object of the present invention relates to the provision of methods for determining female fertility potential.

In particular, it is an object of the present invention to provide a method to improve female fertility potential and/or treat female infertility.

Thus, one aspect of the invention relates to a method for predicting the fertility potential of a female subject, the method comprising
determining the level of RANKL and/or OPG in a sample of follicular fluid or blood from the female subject;
comparing said level(s) to one or more corresponding reference levels; and
predicting that said subject is unlikely to have normal fertility potential (low fertility potential) when:
   said RANKL level is below said corresponding reference level; and/or
   said OPG is above said corresponding reference level(s);

### OR

predicting that said subject is likely to have a normal fertility potential when:
   said RANKL level is equal or above said corresponding reference level; and/or
   said OPG level is equal to or below said corresponding reference level.

Another aspect of the present invention relates to a method for monitoring the development of the fertility potential for a female subject, the method comprising
determining a first level of RANKL and/or OPG in a first sample of follicular fluid or blood from the female subject;
determining a second level of RANKL and/or OPG in a corresponding second sample from the female subject, wherein the second sample has been obtained at a later time point than the first sample;
comparing the corresponding levels in the first and the second sample;
   wherein
a lower RANKL level in the second sample compared to the first sample is indicative of a worsened fertility potential; and/or
a higher OPG level in the second sample compared to the first sample is indicative of a worsened fertility potential;
equal RANKL and/or OPG levels in the first and second sample is indicative of an unchanged fertility potential; and
a higher RANKL level in the second sample compared to the first sample is indicative of an improved fertility potential; and/or
a lower OPG level in the second sample compared to the first sample is indicative of an improved fertility potential.

Yet another aspect of the present invention is to provide to a compound for use in treatment and/or improvement and/or prevention of female infertility in a mammal and/or for use in improving female fertility potential;

wherein said compound is
a stimulator of (ovarian) RANKL expression;
an agonist or inducer/activator of RANKL, such as inducer/activator of RANKL binding to RANK;
an antagonist or inhibitor of osteoprotegerin (OPG);
RANKL; and/or
an inhibitor of RANKL, such as an inhibitor, which inhibits the binding of RANK to RANKL.

Example 4 shows that administration of the well-characterized RANKL inducer PTH had a beneficial effect on fertility in mice.

Example 5 shows that administration of the well characterized RANKL inhibitor OPG of (uterine) RANKL expression ad a beneficial effect on fertility in mice as OPG levels in follicular fluid is 100 fold higher and not influenced by OPG treatment. Thus, in an embodiment, the compound is for treatment and/or improvement of implantation of the oocyte into the uterus in healthy and/or infertile female subjects;

wherein said compound is an inhibitor of uterine RANKL signaling (such as OPG) and thereby improve implantation of the oocyte and secures more healthy pregnancies. As outlined in example 5, OPG treatment improves fertility in vivo, but does not change ovarian weight, likely due to low serum and high follicular fluid OPG prior to treatment.

Still another aspect of the present invention is to provide an in vitro method for determining the chances for an egg to be successfully fertilized and/or to lead to a successful pregnancy (child birth), the method comprising
determining, in vitro, in a sample comprising at least one unfertilized egg, preferably only one egg, the level of RANKL and/or OPG;
comparing said level(s) to one or more corresponding reference levels; and
predicting that said one or more eggs are unlikely of being successfully fertilized and/or to lead to a successful pregnancy after fertilization when:
   said RANKL level is below said corresponding reference level; and/or
   said OPG is above said corresponding reference level(s);

### OR

predicting that said one or more eggs are likely to be successfully fertilized and/or to lead to a successful pregnancy after fertilization when:
   said RANKL level is equal or above said corresponding reference level; and/or
   said OPG level is equal to or below said corresponding reference level.

A further aspect of the invention relates to an in vitro method for improving changes for an egg to be successfully fertilized and/or to lead to a successful pregnancy, the method comprising administering, ex vivo, to an unfertilized egg, a compound according to the invention.

In an embodiment, said compound is
a stimulator of (ovarian) RANKL expression;
an agonist or inducer/activator of RANKL, such as inducer/activator of RANKL binding to RANK;
an antagonist or inhibitor of osteoprotegerin (OPG); and/or
RANKL;
An inhibitor of RANKL, such as an inhibitor, which inhibits the binding of RANK to RANKL.

### BRIEF DESCRIPTION OF THE FIGURES

FIG. 1 shows ovarian weight (in grams) in wild type mice (n=10) and Mice with RANKL deficiency (n=13). The data is presented as mean+Std. error of mean.

FIG. 2 shows uterus weight in wild type mice (n=10) and mice with RANKL deficiency (n=13). Data presented as mean+Std. error of mean.

FIG. 3 shows litter size in wild type mice (n=10) and Mice with RANKL deficiency (n=13). The data is presented as mean+Std. error of mean.

FIG. 4 shows the average number of dead pups early after birth in wild type mice (n=35) and mice with RANKL deficiency (n=35). Data presented as median +Std. deviation.

FIGS. 5 and 6 show results of RT PCR analysis of Human tissue samples. (FIG. 5): types of tissue tested. (FIG. 6) Expression level of the genes; RANKL-1, RANKL-2, RANK, OPG, NFKB, RELa and RELb in the different tissue samples.

FIG. 7 shows paired data of stable expression levels of both (A) RANKL and OPG (B), measured twice in the same woman, from two consecutive cycles of IVF/ICSI (paired T-test not significant). (C) Amount of serum RANKL and follicular RANKL, which are within the same range, but not correlated.

FIG. 8 shows statistically significant linear regression of follicular- and serum hormones. (A) inverse association between follicular OPG and sRANKL, (B) positive association between follicular sRANK and follicular AMH, (C) inverse association between follicular OPG and AMH, (D) inverse association between serum sRANKL and follicular SHBG, (E) inverse association between serum sRANKL and serum SHBG, (F) inverse association between serum sRANKL and serum estradiol.

FIG. 9 shows follicular or serum RANKL and OPG in relation to age. (A-B) follicular sRANKL declines with age, shown in linear regression (A) and grouped analysis (B) in a non-parametric comparison of groups, Kruskal-Wallis, (C-D) follicular OPG increases with age, shown in linear regression (C) and grouped analysis (D), (E-F) serum sRANKL declines with age, shown in linear regression (E) and grouped analysis (F).

FIG. 10 shows association between follicular RANKL or OPG and follicular number (A-B) follicular sRANKL increases with number of follicles naturally matured by the woman (sum of left and right ovary), shown in linear regression (A) and grouped analysis (B) in a non-parametric comparison of groups, Kruskal-Wallis, (C-D) follicular OPG decreases with number of follicles naturally matured by the woman (sum of left and right ovary), shown in linear regression (C) and grouped analysis (D).

FIG. 11 shows a comparison of healthy and infertile women. Grouped analyses showing: (A) healthy women have higher follicular sRANKL levels than infertile women, (B) but comparable follicular OPG levels.

FIG. 12 shows RANKL levels related to pregnancy and birth. (A) grouped analyses showing borderline-higher serum sRANKL in women who become pregnant after ICIS/IVF and carry the child to term, compared to women who have early spontaneous miscarriage, (B) grouped analyses showing significantly higher serum sRANKL in women who become pregnant after ICIS/IVF and carry the fetus to week seven with ultrasonic verified heartbeat, compared to women who have early spontaneous miscarriage.

FIG. 13 shows a linear regression between SHBG and birth size. (A) Showing declining birth weight and (B) declining birth length with increasing levels of maternal follicular SHBG levels at the time of conception.

FIG. 14 shows fertility study of mice pared with WT males with or without treatment with PTH. (A) Pregnancies. (B): Total number of pups. (C): Pups per female.

FIG. 15 shows fertility study of mice pared with WT males with or without treatment with OPG. (A) Pregnancies. Top right: Total number of pups. Bottom left: Pups per female.

FIG. 16 shows a Spearmann correlation between serum OPG and follicular OPG.

FIG. 17 shows follicular fluid 1,25D3 levels in women obtaining pregnancies, validated by ultrasound after IVF compared with women having no pregnancy.

The present invention will now be described in more detail in the following.

### DETAILED DESCRIPTION OF THE INVENTION

### Definitions

Prior to discussing the present invention in further details, the following terms and conventions will first be defined.

Antibody

Antibodies of the invention include polyclonal, monospecific polyclonal, monoclonal, recombinant, chimeric, humanized, fully human, single chain and/or bispecific antibodies including antibody fragments. Examples of such fragments include Fab F(ab′), F(ab)′, Fv, and sFv fragments. The antibodies may be generated by enzymatic cleavage of full-length antibodies or by recombinant DNA techniques, such as expression of recombinant plasmids containing nucleic acid sequences encoding antibody variable regions.

Polyclonal antibodies are heterogeneous populations of antibody molecules derived from the sera of animals immunized with an antigen. An antigen is a molecule or a portion of a molecule capable of being bound by an antibody, which is additionally capable of inducing an animal to produce antibody capable of binding to an epitope of that antigen. An antigen can have one or more epitopes. The specific reaction referred to above is meant to indicate that the antigen will react, in a highly selective manner, with its corresponding antibody and not with the multitude of other antibodies, which can be evoked by other antigens.

Monoclonal antibodies (mAbs) contain a substantially homogeneous population of antibodies specific to antigens, which population contains substantially similar epitope binding sites. Such antibodies may be of any immunoglobulin class including IgG, IgM, IgE, IgA, IgD and any subclass thereof. A hybridoma producing a monoclonal antibody of the present invention may be cultivated in vitro, in situ, or in vivo. Production of high titers in vivo or in situ is a preferred method of production.

Examples of suitable methods for preparing monoclonal antibodies include hybridoma methods of Kohler et al., Nature 256, 495-497 (1975), and the human B-cell hybridoma method, Kozbor, J. Immunol. 133, 3001 (1984); Brodeur et al., Monoclonal Antibody Production Techniques and Applications, pp. 51-63 (Marcel Dekker, Inc., New York, 1987); and Harlow and Lane, Antibodies: A Laboratory Manual, Cold Spring Harbor Laboratory (1988); the contents of which references are incorporated entirely herein by reference.

A particularly preferred method for producing monoclonal antibodies is the XenoMouse as described in Green, LL, J. Immunol. Methods (1999), Vol. 231, 11-25, with an OPGbp/RANKL peptide, such as a full-length human RANKL protein.

Preferred methods for determining monoclonal antibody specificity and affinity by competitive inhibition can be found in Harlow et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc and Wiley Interscience, N.Y., (1992, 1993), and Muller, Meth. Enzymol., 92:589-601 (1983).

Chimeric antibodies are molecules in which different portions are derived from different animal species, such as those having a variable region derived from a murine monoclonal antibody and a human immunoglobulin constant region.

The term “chimeric antibody”, as used herein, includes monovalent, divalent or polyvalent immunoglobulins. A monovalent chimeric antibody is a dimer (HL) formed by a chimeric H chain associated through disulfide bridges with a chimeric L chain. A divalent chimeric antibody is tetramer (H2L2) formed by two HL dimers associated through at least one disulfide bridge. A polyvalent chimeric antibody can also be produced, for example, by employing a CH region that aggregates (e.g., from an IgM H chain, or [micro] chain).

Murine and chimeric antibodies, fragments and regions of the present invention may comprise individual heavy (H) and/or light (L) immunoglobulin chains.

Selective binding agents, such as antibodies, fragments, or derivatives, having chimeric H chains and L chains of the same or different variable region binding specificity, can also be prepared by appropriate association of the individual polypeptide chains, according to known method steps, e.g., according to Ausubel et al., eds. Current Protocols in Molecular Biology, Wiley Interscience, N.Y. (1993), and Harlow et al., Antibodies: A Laboratory Manual, Cold Spring Harbor Laboratory Press, Cold Spring Harbor, N.Y. (1988). The contents of these references are incorporated entirely herein by reference. With this approach, hosts expressing chimeric H chains (or their derivatives) are separately cultured from hosts expressing chimeric L chains (or their derivatives), and the immunoglobulin chains are separately recovered and then associated. Alternatively, the hosts can be co-cultured and the chains allowed to associate spontaneously in the culture medium, followed by recovery of the assembled immunoglobulin, fragment or derivative.

Antigen Binding Domain

The term “antigen binding domain” or “antigen binding region” or “fragment or derivative thereof” refers to that portion of the selective binding agent (such as an antibody molecule) which contains the amino acid residues that interact with an antigen and confer on the binding agent its specificity and affinity for the antigen. Preferably, the antigen binding region will be of human origin. In other embodiments, the antigen binding region can be derived from other animal species, in particular domestic animal and rodents such as rabbit, rat or hamster.

Effective Amount

The terms “effective amount” and “therapeutically effective amount”, refer to an amount of a selective binding agent that is useful or necessary to support an observable change in the level of one or more biological activities, wherein said change may be either an increase or decrease.

Expression Level

The “expression level” or “level” as used herein refers to the absolute or relative amount of protein in a given sample. Thus, the expression level refers to the amount of protein in a sample. The expression level is usually detected using conventional detection methods.

In another preferred embodiment, the expression level refers to the total protein level of the protein in question in a semen sample.

Female Fertility/Female Fertility Potential

Infertility is a prevalent condition and the cause may be female, male, a combination, or unexplained. 12 months of unprotected intercourse without contraception and without conception is defined as infertility.

Female Subject

Reference to a “female subject” or “subject” or an “individual” includes a human or non-human species of mammals including primates, livestock animals (e.g. sheep, cows, pigs, horses, donkey, goats), laboratory test animals (e.g. mice, rats, rabbits, guinea pigs, hamsters) and companion animals (e.g. dogs, cats). The present invention has applicability, therefore, in human medicine as well as having livestock and veterinary and wild life applications. In a preferred embodiment, the mammal is a human. In a particular preferred embodiment, the mammal is a woman.

OPG

Osteoprotegerin (OPG), also known as osteoclastogenesis inhibitory factor (OCIF) or tumour necrosis factor receptor superfamily member 11B (TNFRSF11B), is a cytokine receptor of the tumour necrosis factor (TNF) receptor superfamily encoded by the TNFRSF11B gene.

RANK

Receptor activator of nuclear factor κ B (RANK), also known as TRANCE receptor or TNFRSF11A, is a member of the tumor necrosis factor receptor (TNFR) molecular sub-family. RANK is the receptor for RANK-Ligand (RANKL) and part of the RANK/RANKL/OPG signaling pathway that regulates osteoclast differentiation and activation. It is associated with bone remodeling and repair, immune cell function, lymph node development, thermal regulation, and mammary gland development. Osteoprotegerin (OPG) is a decoy receptor for RANK, and regulates the stimulation of the RANK signaling pathway by competing for RANKL. The cytoplasmic domain of RANK binds TRAFs 1, 2, 3, 5, and 6 which transmit signals to downstream targets such as NF-κB and JNK.

RANKL

Receptor activator of nuclear factor kappa-B ligand (RANKL), also known as tumor necrosis factor ligand superfamily member 11 (TNFSF11), TNF-related activation-induced cytokine (TRANCE), osteoprotegerin ligand (OPGL), and osteoclast differentiation factor (ODF), is a protein that in humans is encoded by the TNFSF11 gene.

RANKL is known as a type II membrane protein and is a member of the tumor necrosis factor (TNF) superfamily. RANKL has been identified to affect the immune system and control bone regeneration and remodeling. RANKL is an apoptosis regulator gene, a binding partner of osteoprotegerin (OPG), a ligand for the receptor RANK and controls cell proliferation by modifying protein levels of Id4, Id2 and cyclin Dl.

RANKL is expressed in several tissues and organs including: skeletal muscle, thymus, liver, colon, small intestine, adrenal gland, osteoblast, mammary gland epithelial cells, prostate and pancreas. Variation in concentration levels of RANKL throughout several organs reconfirms the importance of RANKL in tissue growth (particularly bone growth) and immune functions within the body.

Studies in human testis and vitamin D receptor (VDR) knock-out mice have shown that several bone factors such as Runx2, osterix, and osteocalcin are expressed in the normal testis and testicular cancer. One of these factors is the receptor activator of NF-κB ligand (RANKL). The RANKL system is a powerful regulator of bone resorption that comprises three components: RANKL, a transmembrane ligand that following binding to the receptor RANK on a neighbouring cell subsequently activates NF-κB and regulates cell cycle i.e. proliferation, differentiation and apoptosis. The transmembrane RANKL protein resides in osteocytes and activates RANK in immature osteoclasts, which induces osteoclastogenesis and promotes bone resorption. Osteoprotegerin (OPG) is an endogenous secreted decoy receptor that binds RANKL and blocks its signaling thereby preventing osteoclast differentiation and activation. RANKL can also be found in circulation, suggesting a putative endocrine function of the protein. Indeed, recently novel extra-skeletal functions of RANKL have been proposed including regulation of glucose homeostasis.

In the present context, the terms “soluble RANKL” or “sRANKL” refer to the free fraction of RANKL. sRANKL is not bound to OPG.

Reference Level

In the context of the present invention, the term “reference level” relates to a standard in relation to a quantity, which other values or characteristics can be compared to.

A reference level can be average values from one or more healthy subjects (with normal female fertility potential), or actually also from values from one or more subjects with reduced fertility potential. A reference level could also be from one or more samples from the same subject obtained at previous time points. Again, the exact level of the reference level depends on the desired sensitivity and specificity. The clinician can decide on that. In the example section different determined values for RANKL and OPG are provided.

Risk Assessment

The present inventors have successfully developed a new method to predict the risk of a female subject to be infertile. To determine whether a patient has an increased risk of being infertile a cut-off (reference level) must be established. This cut-off may be established by the laboratory, the physician or on a case-by-case basis for each patient.

The cut-off level could be established using a number of methods, including: multivariate statistical tests (such as partial least squares discriminant analysis (PLS-DA), random forest, support vector machine, etc.), percentiles, mean plus or minus standard deviation(s); median value; fold changes.

The multivariate discriminant analysis and other risk assessments can be performed on the free or commercially available computer statistical packages (SAS, SPSS, Matlab, R, etc.) or other statistical software packages or screening software known to those skilled in the art.

As obvious to one skilled in the art, in any of the embodiments discussed above, changing the risk cut-off level could change the results of the discriminant analysis for each subject.

Statistics enables evaluation of the significance of each level. Commonly used statistical tests applied to a data set include t-test, f-test or even more advanced tests and methods of comparing data. Using such a test or method enables the determination of whether two or more samples are significantly different or not.

The significance may be determined by the standard statistical methodology known by the person skilled in the art.

The chosen reference level may be changed depending on the mammal/subject for which the test is applied.

Preferably, the subject according to the invention is a human subject, such as a male subject considered at risk of being infertile.

The chosen reference level may be changed if desired to give a different specificity or sensitivity as known in the art. Sensitivity and specificity are widely used statistics to describe and quantify how good and reliable a biomarker or a diagnostic test is. Sensitivity evaluates how good a biomarker or a diagnostic test is at detecting a disease, while specificity estimates how likely an individual (i.e. control, patient without disease) can be correctly identified as not at risk. Several terms are used along with the description of sensitivity and specificity; true positives (TP), true negatives (TN), false negatives (FN) and false positives (FP). If a disease is proven to be present in a sick patient, the result of the diagnostic test is considered to be TP. If a disease is not present in an individual (i.e. control, patient without disease), and the diagnostic test confirms the absence of disease, the test result is TN. If the diagnostic test indicates the presence of disease in an individual with no such disease, the test result is FP.

Finally, if the diagnostic test indicates no presence of disease in a patient with disease, the test result is FN.

Sensitivity

As used herein the sensitivity refers to the measures of the proportion of actual positives which are correctly identified as such—in analogy with a diagnostic test, i.e. the percentage of mammals or people having a fertility potential below normal who are identified as having a fertility potential below normal.

Usually the sensitivity of a test can be described as the proportion of true positives of the total number with the target disorder i.e. a fertility potential below normal. All patients with the target disorder are the sum of (detected) true positives (TP) and (undetected) false negatives (FN).

Specificity

As used herein the specificity refers to measures of the proportion of negatives which are correctly identified—i.e. the percentage of mammal with a normal fertility potential that are identified as not having a fertility potential below normal. The ideal diagnostic test is a test that has 100% specificity, i.e. only detects mammal with a fertility potential below normal and therefore no false positive results, and 100% sensitivity, and i.e. detects all mammals with a fertility potential below normal and therefore no false negative results.

For any test, there is usually a trade-off between each measure. For example in a manufacturing setting in which one is testing for faults, one may be willing to risk discarding functioning components (low specificity), in order to increase the chance of identifying nearly all faulty components (high sensitivity). This trade-off can be represented graphically using a ROC curve.

Selecting a sensitivity and specificity it is possible to obtain the optimal outcome in a detection method. In determining the discriminating value distinguishing mammals having a fertility potential below normal, the person skilled in the art has to predetermine the level of specificity. The ideal diagnostic test is a test that has 100% specificity, i.e. only detects mammals with a fertility potential below normal and therefore no false positive results, and 100% sensitivity, and i.e. detects all mammals with a fertility potential below normal and therefore no false negative results. However, due to biological diversity no method can be expected to have 100% sensitive without including a substantial number of false negative results.

The chosen specificity determines the percentage of false positive cases that can be accepted in a given study/population and by a given institution. By decreasing specificity an increase in sensitivity is achieved. One example is a specificity of 95% that will result in a 5% rate of false positive cases. With a given prevalence of 1% of e.g. a fertility potential below normal in a screening population, a 95% specificity means that 5 individuals will undergo further physical examination in order to detect one (1) fertility potential below normal if the sensitivity of the test is 100%.

As will be generally understood by those skilled in the art, methods for screening for fertility potentials are processes of decision making and therefore the chosen specificity and sensitivity depends on what is considered to be the optimal outcome by a given institution/clinical personnel.

### ABBREVIATIONS

1.250H2D3: The active form of Vitamin D
FSH: Follitropin
IL-1: Interleukin 1
LH/hCG: Lutropin/Choriogonadotropin
PTH: Parathyroidea hormone
PTHRP: Parathyroidea hormone-related protein
SHBG: Sex hormone binding globulin
TNF: Tumor necrosis factors
RANKL: Receptor activator of nuclear factor kappa-B ligand
OPG: Osteoprotegerin
RANK: Receptor activator of nuclear factor kappa-B
SIK: Salt inducible Kinases 1-3

Predicting Fertility Potential in a Female Subject

As also outlined above, the present invention relates to the identification that the RANKL/OPG/RANK system is present in the female reproductive system, and that RANKL and OPG levels may be indicative of the female fertility potential (see example 1 (mice data) and examples 2-3 (human data). Thus, a first aspect of the invention relates to a method for predicting the fertility potential of a female subject, the method comprising
determining the level of RANKL and/or OPG in a sample of follicular fluid or blood from the female subject;
comparing said level(s) to one or more corresponding reference levels; and
predicting that said subject is unlikely to have normal fertility potential (low fertility potential) when:
   said RANKL level is below said corresponding reference level; and/or
   said OPG is above said corresponding reference level(s);

### OR

predicting that said subject is likely to have a normal fertility potential when:
   said RANKL level is equal or above said corresponding reference level; and/or
   said OPG level is equal to or below said corresponding reference level.

In the present context, the term “unlikely to have normal fertility potential” refers to the subject having a low fertility potential or even being infertile.

In an embodiment, the level of RANKL is determined, preferably the level of sRANKL. Soluble RANKL (sRANKL) may be determined.

RANKL levels may be determined in different ways. Thus, in an embodiment, the level is the protein level, such as protein level of RANKL and/or OPG, such as soluble RANKL.

As outlined above, RANKL and/or OPG may be determined in different sample types. In an embodiment, the level of RANKL and/or OPG is determined in follicular fluid. In example 3, data from human follicular fluid is provided.

In an embodiment, the RANKL/OPG ratio is determined. In such case a high ratio (above a reference level) will be indicative of said subject is likely to have a normal fertility potential and a low ratio (below or equal to a reference level) will be indicative of said subject is likely to have a low fertility potential. Thus, ratios between the different markers by improve the statistical power of the method.

In yet an embodiment, said determination of RANKL and/or OPG is determined at the protein level by a method selected from the group consisting of immunohistochemistry, immunocytochemistry, FACS, ImageStream, Western Blotting, ELISA, Luminex, Multiplex, Immunoblotting, TRF-assays, immunochromatographic lateral flow assays, Enzyme Multiplied Immunoassay Techniques, RAST test, Radioimmunoassays, immunofluorescence and immunological dry stick assays, such as a lateral flow assay.

In a preferred embodiment, said determination is performed by ELISA, immunocytochemistry or a lateral flow assay.

The skilled person may apply different reference levels depending on the desired specificity and sensitivity. Thus, in an embodiment, the one or more references levels are selected from the group consisting of:
the range 0.1-0.6 pmol/1 serum sRANKL, such as 0.2-0.5 pmol/I, or such as 0.3-0.4 pmol/l serum sRANKL
above 0.2 pmol/1 serum sRANKL, such as above 0.3 pmol/1 or above 0.4 pmol/1 serum sRANKL;
below 0.6 pmol/1 serum sRANKL, such as below 0.5 pmol/I, or below 0.4 pmol/I, or below 0.3 serum sRANKL;
the range 1-6 pmol/1 serum OPG, such as in the range 2-5 pmol/I, or such as 3-4 pmol/1 serum OPG;
above 1 pmol/1 serum OPG, such as above 2 pmol/1 serum OPG, such as above 3 pmol/1 serum OPG, such as above 4 pmol/1 serum OPG, or such as above 5 pmol/1 serum OPG;
below 6 pmol/1 serum OPG, such as below 5 pmol/1 serum OPG, such as below 4 pmol/1 serum OPG, such as below 3 pmol/1 serum OPG, or such as below 2 pmol/1 serum OPG;
the range 0-2 pmol/l follicular sRANKL, such as 0.1-2 pmol/l follicular sRANKL, such as 0.5-2 pmol/l follicular sRANKL, such as 0.2-1.8 pmol/1 follicular sRANKL, such as 0.5-1.5 pmol/l follicular sRANKL, or such as 0.8-1.3 pmol/1 follicular sRANKL;
above 0.01 pmol/l follicular sRANKL, such as above 0.03 pmol/l follicular sRANKL, such as above 0.1 pmol/l follicular sRANKL, such as above 0.5 pmol/1 follicular sRANKL, or such as above 1 pmol/1 follicular sRANKL,
below 2 pmol/1 follicular sRANKL, such as below 1.5 pmol/l follicular sRANKL, such as below 1 pmol/l follicular sRANKL, such as below 0.8 pmol/1 follicular sRANKL, or such as below 0.5 pmol/1 follicular sRANKL;
the range 100-1000 pmol/1 follicular OPG, such as in the range 200-900 pmol/1 follicular OPG, such as in the range 300-700 pmol/l follicular OPG or such as in the range 400-600 pmol/l follicular OPG;
above 100 pmol/1 follicular OPG, such as above 100 pmol/l follicular OPG, such as above 300 pmol/1 follicular OPG, such as above 500 pmol/l follicular OPG, or such as above 700 pmol/l follicular OPG,
below 1000 pmol/l follicular OPG, such as below 800 pmol/l follicular OPG, such as below 600 pmol/l follicular OPG, such as below 500 pmol/l follicular OPG, or such as below 300 pmol/l follicular OPG.

The female fertility potential may depend on one or more different factors. Thus, in an embodiment, the female fertility potential includes one or more indications/factors selected from the group consisting of ovarian function, ovarian follicle number, number of mature follicles, ability to mature follicles, ability for the female to carry a child to life birth, reduction in risk of abortions, normal endometrial and uterus function.

AMH is a known factor to evaluate when determining female fertility potential. Anti-Mullerian Hormone (AMH) is produced by the egg cells as the eggs sacs or follicles develop. The AMH levels in the woman's blood is one of the best current indicators of the current quality of her ovarian reserve. Thus, in an embodiment, the method further comprises determining the level of AMH.

In yet an aspect the method further comprises, if said subject is predicted to have low fertility potential, recommending said subject for an assisted reproductive technique (ART), In vitro fertilization) (IVF) and Intracytoplasmic Sperm Injection (ICSI), or recommending said subject (or starting for said subject) a treatment according to the present invention.

In another aspect, the present invention relates to the Use of RANKL and/or OPG as a predictive marker for fertility potential for a female subject.

Monitoring the Development of the Fertility Potential Over Time

As outlined above, using the method of the invention, the fertility potential of a woman can be determined at a given time point. However, the method of the invention may also find used to monitor the fertility potential over time. Thus, an aspect of the invention relates to a method for monitoring the development of the fertility potential for a female subject, the method comprising
determining a first level of RANKL and/or OPG in a first sample of follicular fluid or blood from the female subject;
determining a second level of RANKL and/or OPG in a corresponding second sample from the female subject, wherein the second sample has been obtained at a later time point than the first sample;
comparing the corresponding levels in the first and the second sample;
   wherein
a lower RANKL level in the second sample compared to the first sample is indicative of a worsened fertility potential; and/or
a higher OPG level in the second sample compared to the first sample is indicative of a worsened fertility potential;
equal RANKL and/or OPG levels in the first and second sample is indicative of an unchanged fertility potential; and
a higher RANKL level in the second sample compared to the first sample is indicative of an improved fertility potential; and/or
a lower OPG level in the second sample compared to the first sample is indicative of an improved fertility potential.

In a preferred embodiment, at least the level of RANKL is determined in the first and second sample.

In yet an embodiment, the first and the second sample is follicular fluid and/or blood serum. In a related embodiment, the blood sample is a whole blood sample, a blood serum sample or a blood plasma sample, preferably a blood serum sample.

It may also be advantageous to be able to monitor if a treatment for improving female fertility potential is efficient. Thus, in an embodiment, a treatment for improving the female fertility potential of the female subject has taken place between the sampling of the first and the second sample. It is of course to be understood that additional samples may be take over time to have a continues monitoring of woman.

In an embodiment, the treatment for improving the female fertility potential is selected from group comprising or consisting of hormonal hyperstimulation with combinations of gonadotropins, hCG and assisted reproductive techniques such as IVF, ICSI and inseminations.

In yet an embodiment, the treatment for improving the female fertility potential is a compound for use according to the present invention.

In yet another embodiment, the treatment for improving the female fertility potential is a change of life style, such as a change of diet such as intake of vitamins and minerals, exercise, weight loss, stopping smoking or reduction of smoking or combinations thereof.

It is to be understood that the female subject according to the present invention may be a human or an animal, such as a mammal. Thus, in an embodiment of the present invention the mammal is selected from the group consisting of human, pig, cattle, zebu, donkey, horse, dog, cat, goat, and sheep, preferably a human.

A Compound for Use in Treatment and/or Prevention of Female Infertility

As outlined above, RANKL and OPG are considered biomarkers for the fertility potential of a female subject. Thus, treatments protocols to improve female fertility potential are also part of the present invention. It is to be understood that improvement of female fertility potential may also be treatment of infertility of a female subject. Thus, yet an aspect of the present invention relates to a compound for use in treatment and/or improvement and/or prevention of female infertility in a mammal and/or for use in improving female fertility potential;

wherein said compound is
a stimulator of (ovarian) RANKL expression;
an agonist or inducer/activator of RANKL, such as inducer/activator of RANKL binding to RANK;
an antagonist or inhibitor of osteoprotegerin (OPG);
RANKL; and/or
an inhibitor of RANKL, such as an inhibitor which inhibits the binding of RANK to RANKL.

In an embodiment, the compound is for use in a woman with ovarian failure, such as Polycystic ovarian syndrome (where the woman may have to many follicles).

In another embodiment, the compound is for use in a woman, who has suffered multiple abortions and/or missed pregnancies such as due to impaired implantation or early abortions, and/or polycystic ovarian syndrome.

In an embodiment, multiple abortions are considered more than one abortions, such as more than 2 or 3 abortions, such as 2-10 abortions, such as 3-10, such as 4-10 or 5-10 abortions.

In the present context, the term “missed pregnancies” are considered as no biochemical pregnancy after three or more tranferrals with fertilized oocytes during IVF or ICSI procedures, such as 3-10, such as 4-10, such as 5-10 tranferrals of fertilized oocytes without signs of pregnancy.

In a different embodiment, RANKL stimulators (such as PTH) are for use in woman having
primary ovarian failure; and/or
old age just before menopause, (thus likely only having few oocytes left) and/or
women with low AMH.

In the present context the term “Primary ovarian failure” or “primary ovarian insufficiency” (POI) is to be understood as when a woman's ovaries stop working normally before she is 40. Many women naturally experience reduced fertility when they are about 40 years old. They may start getting irregular menstrual periods as they transition to menopause. For women with POI, irregular periods and reduced fertility start before the age of 40. Sometimes it can start as early as the teenage years. POI is different from premature menopause. With premature menopause, your periods stop before age 40. You can no longer get pregnant. The cause can be natural or it can be a disease, surgery, chemotherapy, or radiation. With POI, some women still have occasional periods. They may even get pregnant. In most cases of POI, the cause is unknown.

In the present context, the term “old age before menopause” (with few oocytes left)” is to be understood as woman age above 35 but before menopause, such as age above 40, above 45 or above 50 but before menopause.

In the present context, the term “women with low AMH” is considered women having a serum AMH level below 25 pmol/I, such as below 20 pmol/I, such as below 10 pmol/I, or such as below 5 pmol/I.

In another embodiment, the compound is for use in treatment of Polycystic ovarian syndrome where the woman has too many follicles;

wherein said compound is an inhibitor of systemic and or follicular RANKL signaling (such as OPG) and thereby reduce maturation of follicles. As outlined in example 5, OPG treatment improves fertility in vivo, but does not change ovarian weight.

In an embodiment, the compound is for use in a woman, who has suffered multiple abortions and/or missed pregnancies such as due to impaired implantation or early abortions, and/or polycystic ovarian syndrome; wherein the compound is an inhibitor of RANKL.

In another embodiment, the woman has primary ovarian failure; and/or old age before menopause, and/or low AMH levels,

wherein the compound is a RANKL stimulator, such as PTH.

As outlined in the example section, since low levels of RANKL and/or high levels of OPG may negatively influence the female fertility potential, the above compounds are considered to be able to improve the fertility potential of a female subject.

In an embodiment, said compound is a stimulator of ovarian RANKL expression. Different compounds may stimulate/enhance RANKL expression. Thus, in a related embodiment, the compound is selected from the group consisting of PTH, PTHRP, vitamin d, 25OHD, 1.250H2D3, 24,250H2D3, Etalpha, IL-1, TNF, FSH, LH, a SIK inhibitor, YKL-05-099 and combinations thereof, preferably the compounds are PTH, PTHrP or a SIK inhibitor.

In another embodiment, the compound is selected from the group consisting of IL-1, IL-6, IL-11, IL-17, TNF-α, vitamin D, Ca²⁺, parathyroid, glucocorticoids, prostaglandin E2, and immunosuppressive drugs.

These compounds are all known to the skilled person to stimulate/enhance RANKL expression.

In yet an embodiment, the compound is an agonist or inducer/activator of RANKL, such as an inducer/activator of RANKL binding to RANK.

In a related embodiment, the compound is LH/hCG or FSH.

In another embodiment, the compound is an antagonist or inhibitor of osteoprotegerin (OPG), such as an antibody inhibiting binding of OPG to RANKL (Nadine et al. Nature Communications volume 10, Article number: 5183 (2019)/WO2018138297) or a peptide comprising the binding site on RANKL for OPG. By inhibiting OPG binding to RANKL, RANKL can bind to RANK, which (without being bound by theory) is considered important for the female fertility potential.

Without being bound by theory, the following considerations can explain the in vivo mice data presented in examples 4 and 5.

Cyt-177-c (OPG-FC)

Without being bound by theory, Cyt-177-c (OPG-FC) is considered an inhibitor of RANKL by inhibiting RANKL-RANK signalling. However, OPG will likely not come into the follicular fluid and exert an effect there because OPG levels in follicular fluid is >100 fold higher than in serum and not influenced by OPG. However, OPG will block RANKL in all extragonadal tissues including the uterus. Short manipulation of the RANKL pathway in all tissues accessibly by an RANKL inhibitor (exemplified by OPG) has no effect on organ weight but increases pregnancies, number of pups and litter size and a tendency towards higher serum AMH. This supports that suppression of RANKL outside the follicle may improve female reproductive function and increase the likelihood of a live birth. The effect on live birth is most likely due to an effect on implantation and survival in the uterus. See also example 5.

Thus, in an embodiment, the compound for use according to the present invention is an inhibitor of RANKL, such as an inhibitor, which inhibits the binding of RANK to RANKL. Thus, in an embodiment, the compound for use is an antagonist or inhibitor, such as antibodies or antigen binding domains (such as Nanobodies or similar), which regulate the interaction between RANKL and RANK, in particular antibodies or antigen binding domains, which block the interaction between RANKL and RANK and/or inhibit at least one activity of RANKL.

In a more specific embodiment, the compound is Denosumab, Cyt-177-c (OPG-FC) or OPG. As outlined in example 5, OPG-FC has been tested in mice as an example of a RANKL inhibitor, since Denosumab does not bind to mice RANKL (Denosumab is specific for human RANKL). EP 3 030 249 B1 discloses a method for treating male infertility using an RANKL-RANK inhibitor such as Denosumab or OPG. It is noted that Denosumab is a well-known antibody, which binds to human RANKL thereby inhibiting RANKL-RANK interaction/signaling.

Thus, in an embodiment the compound for use is Denosumab or compounds with the same or similar binding affinity to RANKL i.e. a biosimilar e.g. in the form of a single domain antibody such as a Nanobody.

However, other known (small molecule) RANKL inhibitors exist. Thus, in an embodiment, the RANKL inhibitor is selected from the group consisting of AS2676293, ABD328, ABD345, SPD-304, and E09241 and active fragments and analogues thereof.
AS2676293 is described in Nakai et al. (Bone Research volume 7, Article number: 1 (2019))
ABD328 and ABD345 are described in Coste etl al. (Ann Rheum Dis. 2015 Jan.; 74(1):220-6).
SPD-304 and analogues of SPD-304 are described in Rinotas et al. (J. Med. Chem. 2020, 63, 20, 12043-12059).
E09241 is described in Han et al. (Front. Pharmacol., 11 Mar. 2019).

In an embodiment, an inhibitor of RANKL-RANK signalling may be for use in treatment/improvement of female infertility, in particular in healthy subjects but also of female subjects suffering from repeated abortions, implantation failure, Polycystic ovarian syndrome and/or endometriosis.

In the present context, the term “endometriosis” is to be understood as a disease of the female reproductive system in which cells similar to those in the endometrium, the layer of tissue that normally covers the inside of the uterus, grow outside the uterus.

PTH

Without being bound by theory, PTH may increase expression of RANKL and inhibit secretion of osteoprotegerin (OPG). Free OPG competitively binds to RANKL as a decoy receptor, preventing RANKL from interacting with RANK, a receptor for RANKL. The binding of RANKL to RANK (facilitated by the decreased amount of OPG available for binding the excess RANKL) may thus stimulate female fertility.

As shown in example 4, short manipulation of the RANKL signaling pathway by injecting of the well-characterized RANKL inducer PTH, had a beneficial effect on fertility in normal mice. PTH treatment doubled pregnancy rates and increased number of pups and number of pups born per female exposed compared with vehicle, which demonstrates that induction of RANKL by PTH prior to and during reproduction is beneficial for live birth rate. The effect could in theory be exerted in many tissues as PTH treatment is not confined for reproductive tissues. However, the significant change in ovary weight indicates that part of the effect is mediated directly in the ovary.

Thus, in an embodiment, an RANKL inducer such as PTH, may be for use in treatment/improvement of female infertility, in particular in healthy subjects but also of female subjects suffering from primary ovarian failure, low AMH levels, and/or old age (likely with low number of oocytes).

Vitamin D (Vitamin D3 (Cholecalciferol)

Without being bound by theory, Vitamin D is considered a stimulant of RANKL by increasing expression of RANKL and maybe even by lowering OPG.

As described in example 4, follicular fluid 1,25D3 levels (another RANKL stimulator) in follicular fluid is higher in women obtaining pregnancies validated by ultrasound after IVF compared with women having no pregnancy.

Thus, in an embodiment, an RANKL inducer such as Vitamin D, may be for use in treatment/improvement of female infertility, in particular in healthy subjects but also of female subjects suffering from primary ovarian failure, low AMH levels, and/or old age (likely with low number of oocytes).

Estrogen/LH/hCG/FSH

These compounds are believed to inhibit gonadal RANKL in male subjects, but to stimulate gonadal RANKL in female subjects.

Antibody Binding to OPG

Antibody binding to OPG, will prevent OPG from inhibiting RANKL-RANK interaction.

Other compounds may also find use. Thus, in yet an embodiment, the compound is selected from the group consisting of imatinib, sex hormones, small molecules.

In an embodiment, the compound is selected from the group consisting of a polyclonal antibody, a monoclonal antibody, a recombinant antibody, a single chain antibody, a bispecific antibody, a nanobody an antibody, wherein the heavy chain and the light chain are connected by a flexible linker, an Fv molecule, an antigen binding fragment, a Fab fragment, a Fab′ fragment, a F(ab′)₂ molecule, a fully human antibody, a humanized antibody, and a chimeric antibody or a fragment or derivative thereof.

Since a desired effect to obtain is to improve the amount of RANKL and/or the activity of RANKL towards RANK, RANKL itself may also considered an effective compound. Thus, in an embodiment, the compound is RANKL or active derivatives or fragments thereof. By active fragments is to be understood fragments able to bind to RANK and initiate the same (or similar effect) as wt RANKL.

This is supported by the data with PTH in Example 4. PTH is a RANKL stimulator. Similarly as explained in example 4, (active) vitamin D (1,25D3) which is also a RANKL stimulator; the concentration of 1,25D3 in follicular fluid correlates with the chance for a woman to conceive a child using IFF.

In an embodiment, said compound is administered in a dosage selected from the group consisting of
1-100 micrograms (mcg) PTH daily dosage, such as 2-800 mcg, such as 2-80 mcg such as 5-60 mcg, such as 10-50 mcg, such as 15-30 mcg, or such as around 20 micrograms (mcg) PTH daily dosage, preferably once a day;
5-800 PTHrP daily dosage, such as 20-500 PTHrP daily dosage, such as 40-200 PTHrP daily dosage, such as 50-150 PTHrP daily dosage, such as 60-120 PTHrP daily dosage or such as around 80 mcg PTHrP daily dosage, preferably once a day;
75-1000 IU of FSH, LH, or hCG daily dosage, such as in the range 100-800, such as in the range 200-800 IU, such as in the range 300-700 IU, or such as in the range 400-600 IU preferably once a day;
500-20000 IU vitamin D daily dosage, such as 1000-10000 IU, such as 2000-8000 IU, such as 4000-6000 IU or such as around 5000 IU vitamin D daily dosage; and
0.25-1.5 mcg Etalpha daily dosage, preferably once a day.

To only obtain the desired effect of the compounds in the desired location, the administration route may be optimized. Thus, in an embodiment, the compound is administered by intrauterine injections, directly into the follicular fluid in the ovary or intra-vaginal.

In sum treatment with a stimulator of follicular RANKL signaling could be by using a known RANKL stimulator as outlined above or by inhibiting OPG that secondary will increase RANKL signaling (as also outlined above) may be used improve fertility in women. Especially during ART (IVF and ICSI) where the women undergoes hyperstimulation and harvest of oocytes to secure an improved outcome of ART by increasing the chance for livebirth and reducing the risk for abortions.

Thus, in an embodiment, said female subject is undergoing ART and/or hyperstimulation, but before harvesting of oocytes.

In another embodiment, the compounds for use are for increasing the chance for livebirth and/or reducing the risk for abortions.

An In Vitro Method for Determining the Changes for an Egg to be Successfully Fertilized and/or to Lead to a Successful Pregnancy

As described above, the method of the invention can be used to determine the fertility potential of a female subject. However, the present invention can also used determine the potential of single egg (or oocyte) (or few eggs) in a sample ex vivo.

When you perform assisted reproduction, you hyperstimulate the woman and more than one follicle will develop. Just before ovulation, all the follicles are aspirated using a small needle to harvest the oocytes. During this process, all the fluid surrounding the oocyte is discarded. Thus, in an aspect the invention relates to measure RANKL and/or OPG in that follicular fluid to identify the best oocytes. If RANKL is higher in the follicular fluid from the other oocyte is the idea that this oocyte will have a bigger chance for becoming a healthy pregnancy than the other oocyte with lower RANKL in the follicular fluid. Thus, RANKL and/or OPG levels in the follicular fluid are used to guide which oocyte to transfer back into the woman after fertilization and culture to 4 cell or blastocyst in vitro. High RANKL indicative for good oocyte quality and thus high pregnancy chance while low OPG is the same.

Thus, an aspect of the invention relates to an in vitro method for determining the chances for an egg to be successfully fertilized and/or to lead to a successful pregnancy (child birth), the method comprising
determining, in vitro, in a sample comprising at least one unfertilized egg (or oocyte), preferably only one egg (or oocyte), the level of RANKL and/or OPG;
comparing said level(s) to one or more corresponding reference levels; and
predicting that said one or more eggs are unlikely of being successfully fertilized and/or to lead to a successful pregnancy after fertilization when:
   said RANKL level is below said corresponding reference level; and/or
   said OPG is above said corresponding reference level(s);

### OR

predicting that said one or more eggs are likely to be successfully fertilized and/or to lead to a successful pregnancy after fertilization when:
   said RANKL level is equal or above said corresponding reference level; and/or
   said OPG level is equal to or below said corresponding reference level.

As outlined above, the reference level can be a general reference level obtained from samples from other women, but it could also be a sample from the same woman, such as from other eggs or oocytes obtained at the same time point. In that way the best egg(s) can be selected to continue with, in the sense that the selected eggs are the ones considered to have the highest chance to progress into a successful pregnancy.

In an embodiment, egg is expected to be fertilized by an assisted reproductive technique (ART), such as selected from the group consisting of In vitro fertilization (IVF) and Intracytoplasmic Sperm Injection (ICSI).

In an embodiment, the sample is taken from the medium holding the one or more eggs.

In Vitro Method for Improving Chances for an Egg to be Successfully Fertilized and/or to Lead to a Successful Pregnancy

The compounds for use according to the invention may also be used in vitro for improving the changes for an egg to be successfully fertilized and/or to lead to a successful pregnancy (after insertion in a woman). Thus, a further aspect of the invention relates to an in vitro method for improving changes for an egg to be successfully fertilized and/or to lead to a successful pregnancy, the method comprising administering, ex vivo, to an unfertilized egg, a compound according to the invention.

In an embodiment, said compound is
a stimulator of (ovarian) RANKL expression;
an agonist or inducer/activator of RANKL, such as inducer/activator of RANKL binding to RANK;
an antagonist or inhibitor of osteoprotegerin (OPG); and/or
RANKL; and/or
An inhibitor of RANKL, such as an inhibitor, which inhibits the binding of RANK to RANKL.

In yet an embodiment, said egg is expected to be fertilized by an assisted reproductive technique (ART), such as selected from the group consisting of In vitro fertilization (IVF) and Intracytoplasmic Sperm Injection (ICSI).

In yet a further embodiment, the compound is administered to or is comprised in the medium holding the egg.

Other Aspects of the Invention

In an aspect the invention relates to a method for treating female infertility in a subject, or for improving fertility potential in female subject, said method comprising administering a therapeutically effective amount of a compound according to the invention.

In an embodiment, said compound is
a stimulator of (ovarian) RANKL expression;
an agonist or inducer/activator of RANKL, such as inducer/activator of RANKL binding to RANK;
an antagonist or inhibitor of osteoprotegerin (OPG); and/or
RANKL; and/or
An inhibitor of RANKL, such as an inhibitor, which inhibits the binding of RANK to RANKL.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

Ovarian function and female reproductive tract is strongly influenced by sex steroids produced by granulosa and Theca cells under the control of luteinizing hormone (LH) and follicle stimulating hormone (FSH), whereas anti-Müllerian hormone (AMH) are produced by the growing follicles stimulated by follicle stimulating hormone (FSH).

There exists an endocrine crosslink between the gonads and bone as sex steroids are potent regulators of skeletal function. Bone specific proteins for instance Osteocalcin and vitamin D has been proposed to stimulate testosterone and gametogenesis. Here we test if some of these putative “endocrine bone factors” may act locally in the female gonad and influence reproduction function. In the following studies, the “endocrine bone factors” are represented by RANKL, RANK and OPG.

### Example 1—Tissue and Cell Specific Function of RANKL In Vivo

Aim of Study

To determine tissue and cell specific function of RANKL-deficiency in vivo.

Material and Methods

Rankl^{fl/fl} mice were obtained (Cat #B6.129-Tnfsf11 from the Jackson Laboratory). We first crossed Rankl^{fl/fl} with VasaCre-Tg mice (Cat #86.FVB-Tg(Ddx4-cre)1Dcas/KnwJ) obtained from the Jackson laboratory. Cre expression was validated by crossing the Rankl^{fl/fl} mice with mice carrying a Rosa26-tdtomato-allele. Our subsequent aims were to generate mice with germ cell specific and global RANKL-deficiency. However, We show that VasaCre;Rankl^{fl/fl} pups had global deletion of RANKL and not exclusively in the germ cells. It is known that offspring of female Vasa-Cre mice will have active Cre globally and this occurs also in 10-15% of pups inheriting the Cre from the father. Genotyping was complicated by interference between presence of the Vasa-Cre allele and genotyping of the floxed Rankl allele. When mice harboring the Vasa-Cre allele were genotyped (primer 1+2) then the Rankl alleles exclusively showed presence of the wildtype alleles. By applying a new primer set that was able to show deletion of Rankl systemically we could generate the expected genotype, which enabled us to control the breeding. We used the primers listed in table 1. The binding site for primer 2 is lost when the floxed allele is deleted, thus primer 1+2 will produce no bands in a homozygous null mouse. Primer 4 was used to detect the original targeted allele prior to deletion of the neo selection cassette. The most likely explanation is that the Vasa-Cre is active in the male germ cells and that sufficient Cre activity remains in the fertilized egg to cause deletion of RANKL in the developing offspring. Therefore, we may be unable to detect the floxed allele because it has been recombined, although this cannot explain why we see a wildtype band. An additional primer set was used and Rank/floxed primers 1+3 produce a 280 bp band for a deleted allele. If this shows up in the tail DNA of the Vasa-Cre-positive mice, then germline deletion has taken place. When we use primer 1+2 or primer 1+3 on pups from the Vasa-Cre male obtained from Jackson that are Rankl wt/wt mated with female Rankl^{fl/fl} then the results were as follows: Using the Rankl floxed 1+2 primers an expected band at 108 bp for the wildtype allele showed and a band at 251 bp for the floxed allele, except for Vasa-Cre positive pups that only show the 108 bp fragment. Primer set 1+3 showed as expected no band in all pups except for a 280 bp band in VasaCre positive pups, which demonstrates global deletion. To validate this global RANKL deletion we breed the mice with wildtype and Rankl^{fl/fl} mice that proved presence of deleted alleles in these mice because RANKL floxed 1+2 showed heterozygotes when breeding with wildtype mice and exclusively the 251 bp band when breeding with Rankl^{fl/fl}. Noteworthy, in 2% of breedings with paternal inherited Vasa-Cre offspring did not have global deletion and became germ cell specific. Primer set 1+3 was able to detect this because it showed no deletion and thus no band at 280 bp. Vasa-Cre activity was validated by crossing the mice with Rosa26-tdtomato-mice and further supported by qPCR and WB from different organs.

TABLE 1
Primers used for genotyping RANKL Sertoli
deficient and global deficient mice

| | | Primer | SEQ |
|---|---|---|---|
| | Primer | sequence | ID |
| Gene | name | 5′-3′ | NO: |
| Rankl | Rankl- | CTGGGAGCGCAGG | 1 |
| (Tnfsf11) | flox 1 | TTAAATA | |
| Rankl | Rankl- | GCCAATAATTAAA | 2 |
| (Tnfsf11) | flox 2 | ATACTGCAGGAAA | |
| Rankl | Rankl- | CTCAGCTTCCAGA | 3 |
| (Tnfsf11) | flox 3 | GGACTGC | |
| Rankl | Rankl- | GTGGGCTCTATGG | 4 |
| (Tnfsf11) | flox 4 | CTTCTGA | |
| Vasa | VasaCre-1 | CACGTGCAGCCGT | 5 |
| | | TTAAGCCGCGT | |
| Vasa | VasaCre-4 | TTCCCATTCTAAA | 6 |
| | | CAACACCCTGAA | |
| Amh | AMHcre-1 | CCTGGAAAATGCT | 7 |
| | | TCTGTCCG | |
| Amh | AMHcre-4 | CAGGGTGTTATAA | 8 |
| | | GCAATCCC | |
| RANKL primer | | | |
| pairs | Amplicon size | | |
| Target | 1 + 2 | 1 + 3 | 4 + 2 |
| Wild type locus | 108 bp | 1278 bp | no product |
| Targeted locus | 1965 bp | 3261 bp | 213 bp |
| Delta neo | 251 bp | 1547 bp | no product |
| Delta exon 3-4 | no | 280 bp | no product |
| | product | | |

Results

To determine the tissue and cell specific function of RANKL inhibition in vivo, RANKL deficient mice were created. We crossed Rankl^{fl/fl} mice with DEAD-Box Helicase 4 (Vasa);Cre Tg mice. Vasa; Cre Tg mice are generally used to create germ cell specific loss. However, Cre activity is global when the allele is either inherited from the mother, or when old males are being used for breeding. Genotyping was performed and further validated by backcrossing the mutant mice with WT mice. This was done by crossing the RANKL-deficient lines with Rosa-reporter mice showing global expression exclusively in the Vasa;Cre positive mice, and Sertoli cell specific activity in Amh;Cre positive mice with no activity in floxed mice. Phenotypic characteristics, including loss of tooth eruption, lactation deficit, and increased bone mass in the Vasa;Cre model were consistent with phenotypic expectations of a global RANKL knockout model (Khosla,S. Minireview: the OPG/RANKL/RANK system. Endocrinology 142, 5050-5055 (2001)). Littermates (Rankl^{fl/fl} mice) without Cre activity were used as control mice. The reproductive phenotype was evaluated at the age of 16-17 weeks.

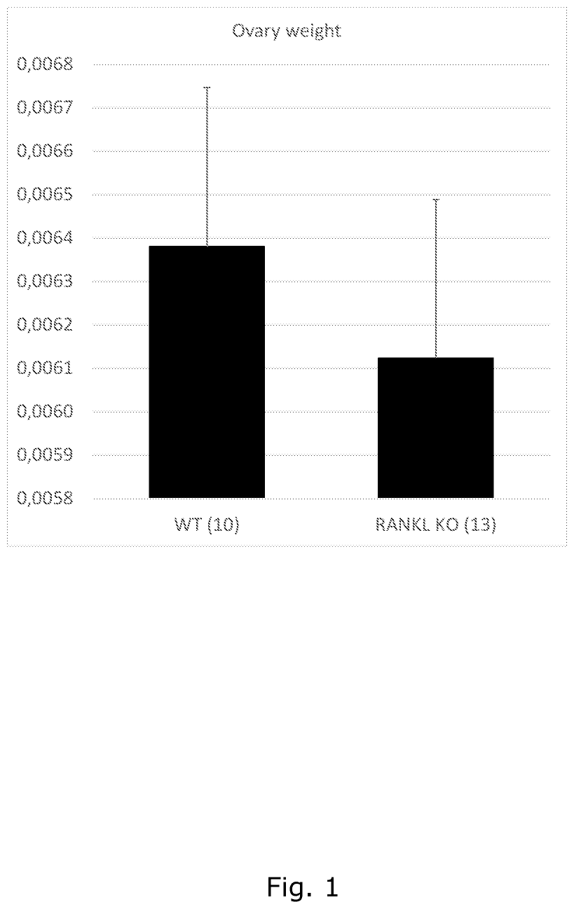

Ovarian weight (in grams) tended to be lower although not statistically significant in global RANKL-deficient compared with Rankl^{fl/fl} mice (FIG. 1).

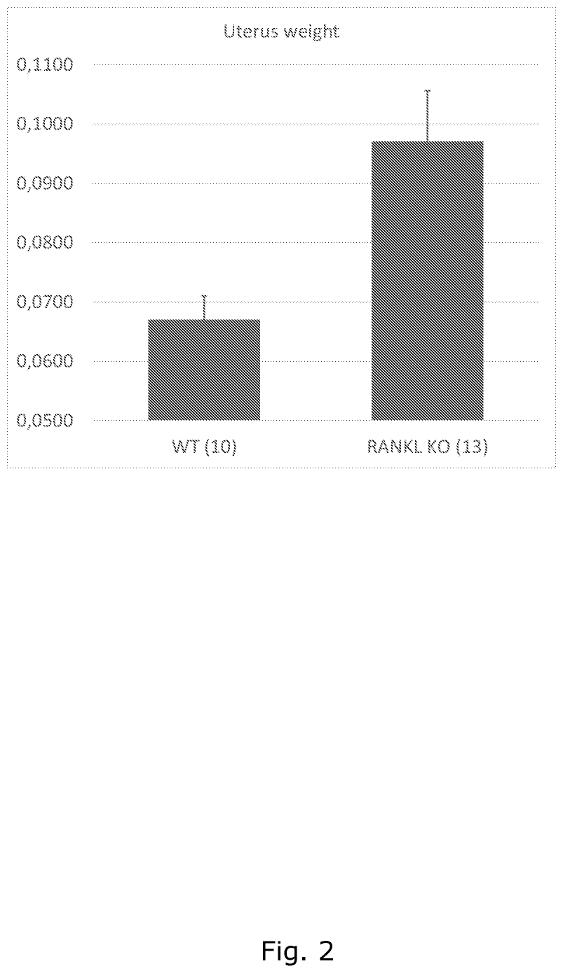

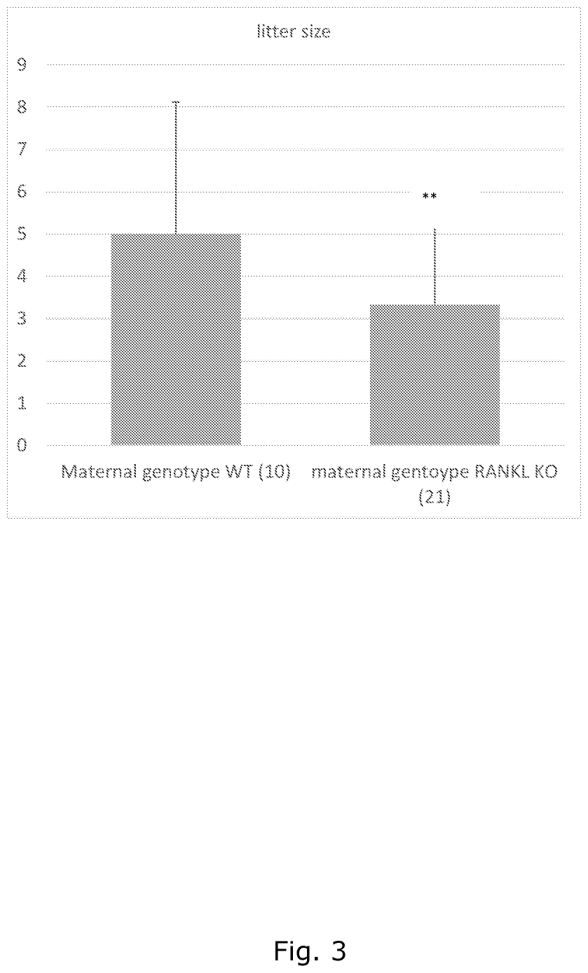

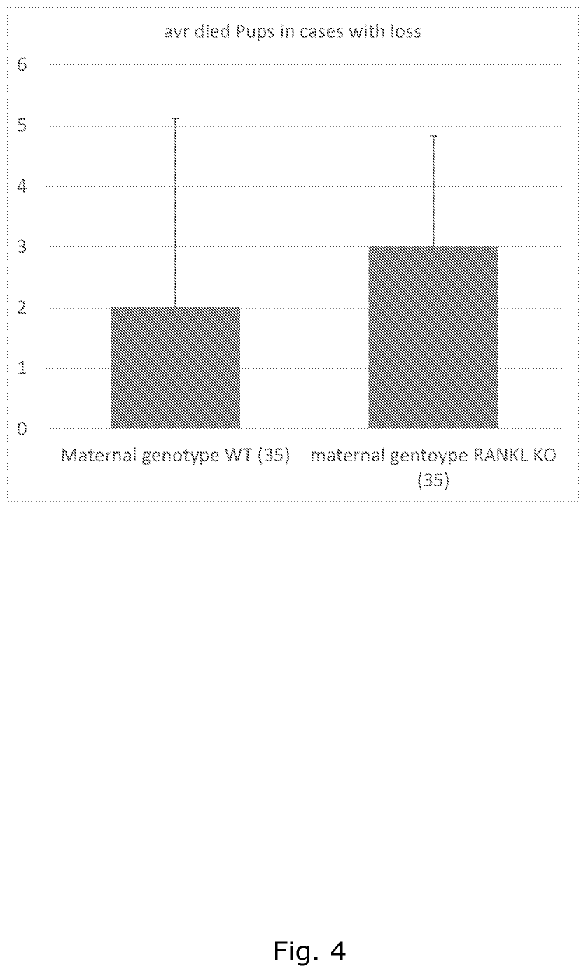

Uterine weight were significantly higher (40% increase) in the RANKL deficient mice compared with Rankl^{fl/fl} mice (FIG. 2). Histologically, fewer follicles were found in the ovary of RANKL-deficient models compared with control mice (data not shown). Mice with global RANKL-deficiency had also lower litter size and significantly more pups dies early after birth even when they had a normal genotype (FIGS. 3 and 4).

Conclusion

The present study demonstrates that the RANKL-system is represented in the female reproductive organs of mice. Further, mice with global suppression of RANKL were found to have increased ovarian weight and fewer follicles in the ovary compared with controls. In addition, mice with global deficiency of RANKL had a lowered female fertility and an increased risk of perinatal death of the fetus. Moreover, the effect on uterus may partially be a direct effect but could also be caused by endocrine changes secondary to the ovarian dysfunction that stimulates uterus growth. These data indicate that RANKL activity and signaling is important for female fertility and successful pregnancies. Further, by using genetic repression of RANKL systemically, we provide insights into the relevance of RANKL as a modifiable regulator of female reproductive function.

In sum, RANKL may be a biomarker for female fertility potential, in the sense that RANKL activity is important for the female fertility potential.

Also, these data make it plausible that by increasing RANKL and/or RANKL activity in a female subject you may improve the fertility potential in that female subject. It is considered that such an improvement may be conducted in vivo but also in vitro during assisted reproductive techniques, such as IVF.

### Example 2—Expression of RANKL, RANK and OPG in Human Ovary

Aim of Study

To compare the expression levels of RANKL, RANK and OPG in different human tissue samples.

Materials and Methods

Material was obtained from department of pathology, Rigshospitalet Denmark in accordance with the Helsinki Declaration after approval from the local ethics committee. Adult ovarian samples were obtained from specimens performed due to suspicion of ovarian cancer and specimens with invasive cancer were discarded. The non-malignant tissue was used for these studies. Tissue fixation and preparation, RNA and cDNA preparation as well as subsequent qRT-PCR and Western blot analysis were performed. All tissues were stained immunohistochemically for RANKL, OPG, and RANK. Briefly, immunohistochemical (IHC) staining was performed according to a standard indirect peroxidase method. All experiments were performed with a negative control staining without the primary antibody. Serial sections and immunofluorescence triple staining were used to examine concomitant expression of RANKL, RANK and OPG as described in detail previously (Jorgensen,A. et al. Nodal Signaling Regulates Germ Cell Development and Establishment of Seminiferous Cords in the Human Fetal Testis. Cell Rep. 25, 1924-1937 (2018)). A detailed description of the antibodies dilutions and retrieval buffers is found in the table below.

| | | Retrieval | | Dilution | |
|---|---|---|---|---|---|
| | | buffer | Dilution | (IF, | |
| Antibody | Dilution | (IHC std./ | (IHC std./ | Human/ | Retrieval |
| designation | (WB) | IHC P.C.) | IHC P.C.) | Mouse | buffer (IF) |
| RANKL C- | 1:200 | CIT/TEG | 1:150/ | — | — |
| term | | | 1:1100 | | |
| RANKL TM | 1:200 | CIT/TEG | 1:250*/ | 1:350 | TEG |
| | | | 1:250 | | |
| RANKL N- | — | CIT/TEG | 1:200/ | 1:500 | TEG |
| term | | | 1:500 | | |
| RANKL | 1:200 | TEG/TEG | —/1:10.000 | — | — |
| extrac. | | | | | |
| RANK C- | 1:200 | CIT/CIT | 1:100/ | — | — |
| term | | | 1:600 | | |
| RANK | | TEG/TEG | 1:200/ | 1:450 | TEG |
| Internal | | | 1:450 | | |
| OPG N- | 1:200 | CIT/TEG | 1:100/ | — | — |
| term | | | 1:600 | | |
| OPG N- | — | CIT/TEG | 1:100/ | 1:400 | TEG |
| term 2 | | | 1:400 | | |

Antibody Dilutions, Retrieval Buffer and Details:

For IHC, antigen retrieval was conducted by microwaving or placing sections in a pressure cooker in indicated retrieval buffer. Citrate buffer: 10 mM, pH 6.0; TEG buffer: 10 mM Tris, 0.5 mM EGTA, pH 9.0. * For staining of spermatozoa antibody sc-9073 was used with TEG buffer at 1:100. Abbreviations: IF, immunofluorescence; IHC, immunohistochemistry; P.C., Pressure Cooker, WB, western blot.

Results

The present study demonstrates that the RANKL-system is present in the female reproductive organs of humans.

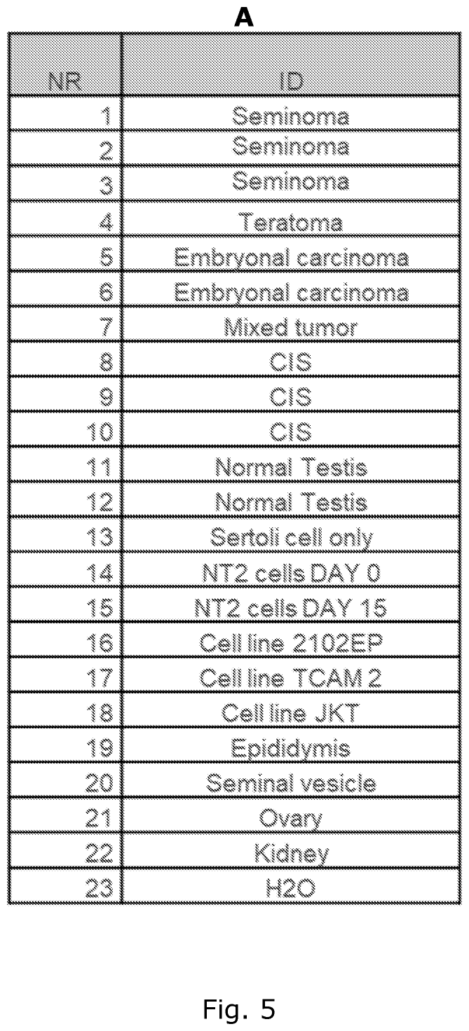

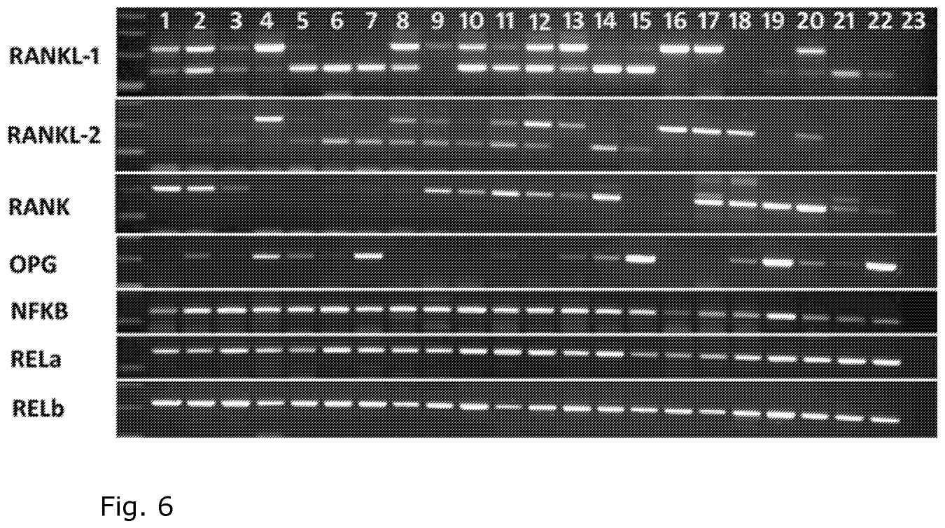

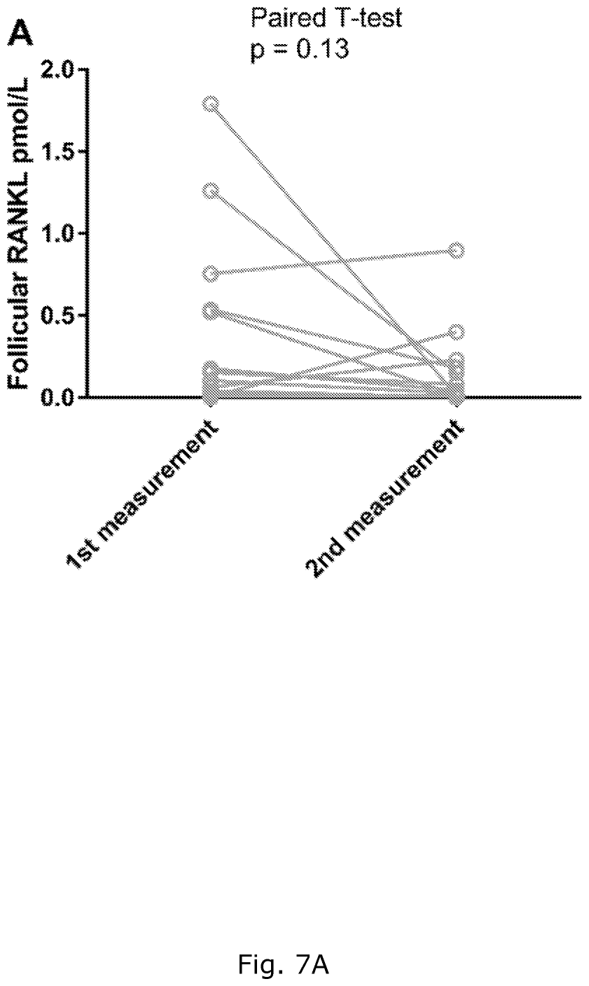

Analysis by RT PCR of different types of female Human tissue samples showed expression of RANKL, RANK, OPG and downstream signaling NFKB, RELa and RELb in human ovary (FIGS. 5 and 6). This was supported by protein expression of RANKL, RANK and OPG that were strongly expressed in the epithelia lining the ovary, the granulosa cells and the cumulus cells surrounding the oocyte (data not shown).

Conclusion

Here, we show that the RANKL/RANK/OPG signalling system and that RANKL, RANK, and OPG, all are expressed in the ovary of humans, suggesting a yet unrecognized regulatory role of female reproductive function.

### Example 3—Association of RANKL and OPG with Female Reproductive Outcome

Aim of Study

To measure RANKL and OPG in serum and follicular fluid surrounding the oocyte in human females and to correlate the RANKL and OPG concentration with reproductive outcome in human females during spontaneous or assisted reproductive techniques.

Materials and Methods

Participants included in the study was infertile couples referred for IUI, IVF or ICSI at the Danish fertility clinic. Both partners should be above 18 years old and the women should be below 43 years old. Women using donor insemination was also included.

The study is a prospective, blinded, single center cohort study. The investigation of all samples will be blinded since investigators have no information about the clinical data and treatment failure/success. All participants was followed until 9 months after their treatment for evaluation of live birth, abortions and malformations, diseases during pregnancy or in the newborns.

All the patients will have finished their initial visit and investigations, before they were invited to participate in the study.

Biochemical Analyses

RANKL and OPG were measured in serum and follicular fluid using two different ELISA's by Biomedica, Austria. Testosterone and estradiol were measured by Access RIA from Coat-a-count, Siemens CV 6% and from Pantex, Santa Monica, USA with a CV of 13%. AMH was measured using Enzyme immunometric assay from Immunotech, Beckman Coulter, USA, CV 11% and finally SHBG with a chemiluminescent immune assay (Access, Beckman Coulter, USA) with CV 8%.

Statistics

Gaussian distribution of all numerical variables was evaluated by distribution in histogram and by plot of residuals to secures validity of performing a regression model. As a result, follicular OPG and follicular RANKL were transformed by natural logarithm when used as dependent in linear regression, whereas serum RANKL was not transformed. All follicular- and sera hormones were analyzed in linear regression. Comparing hormonal levels in groups was done using Mann-Whitney U on untransformed data. Paired data from the same woman was analyzed using a paired T-test.

Follicular and serum hormones that were measured to be below limit of detection were set to (LOD*0.5) in numerical variables.

When analyzing follicular fluid and serum together from the same woman we excluded serum samples that were obtained later than the day of aspiration and more than 16 days prior to aspiration, why 6 serum samples were excluded. Statistical analyses were performed using IBM PASW SPSS version 25 and figures and illustrations were made in GraphPad prism version 7.02.

A total of 289 women were included in the study. 139 women only had one round of ART and the remaining women had between two and six trials.

A total of 544 trials of ART were performed with 341 rounds of IUI, 88 rounds of IVF and 115 rounds of ICSI.

54 pregnancies were obtained (evaluated by increase in urinary- and/or serum-hCG) with 46 showing heart beat in gestational week 7 (evaluated by ultrasound) and finally 37 healthy children were born (one pair of twins). Two children were born slightly preterm in gestational week 35+5 and 36+4 respectively. We collected follicular fluid from 176 aspirations, from 117 different women and measured RANKL in 165 of the samples and OPG in 172 of the samples. We collected 88 serum samples from 74 different women and measured RANKL in 78 samples.

Of the 177 women from whom we collected follicular fluid, 20 women were generally and reproductively healthy with only the man having decreased semen quality and the remaining 157 women had various different gynecological conditions with infertility unspecified, anovulation, PCOS and endometriosis being the most common. Five women acknowledged to be smoking, see baseline table for further characteristics.

| | | Mean | SD (range*) |
|---|---|---|---|
| Women (n = 289) | Age* | 36.6 | 26-44 |
| | BMI* | 23.8 | 18.3-35.4 |
| Follicular fluid (n = 117) | Estradiol (nmol/L) | 1418.4 | 795.2 |
| | Testosterone (nmol/L) | 28.0 | 10.0 |
| | AMH (pmol/L) | 34.2 | 87.4 |
| | SHBG (nmol/L) | 120.3 | 59.5 |
| | RANKL (pmol/L) | 0.073 | 0.234 |
| | OPG (pmol/L) | 219.8 | 192.1 |
| Serum (n = 88) | Estradiol (nmol/L) | 3384.1 | 3543.0 |
| | Testosterone (nmol/L) | 1.59 | 1.02 |
| | AMH (pmol/L) | 20.5 | 22.8 |
| | SHBG (nmol/L) | 115.1 | 64.0 |
| | RANKL (pmol/L) | 0.294 | 0.119 |

Results

RANKL and OPG in Serum and Follicular Fluid

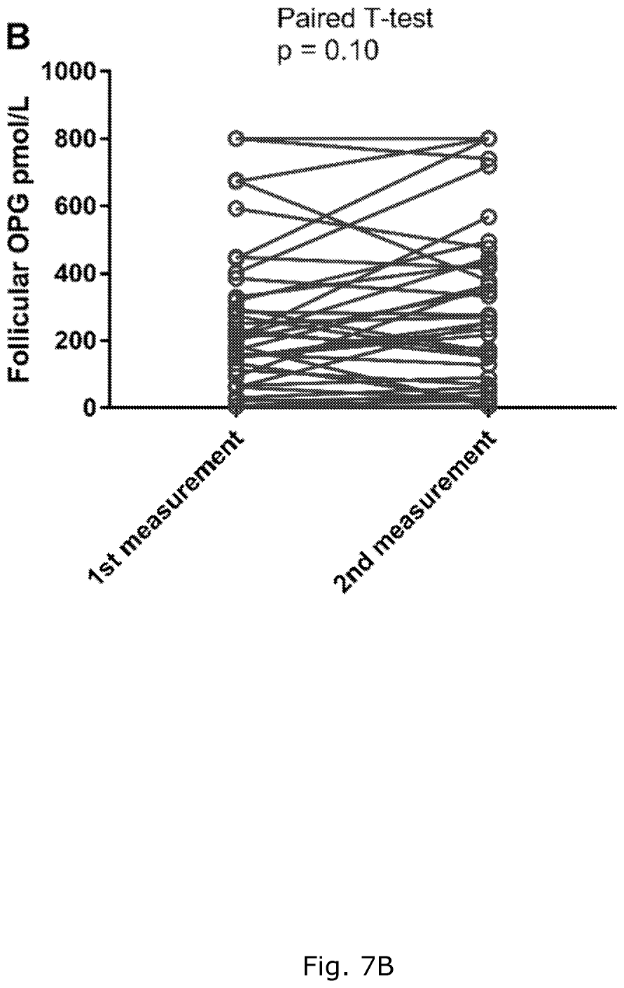

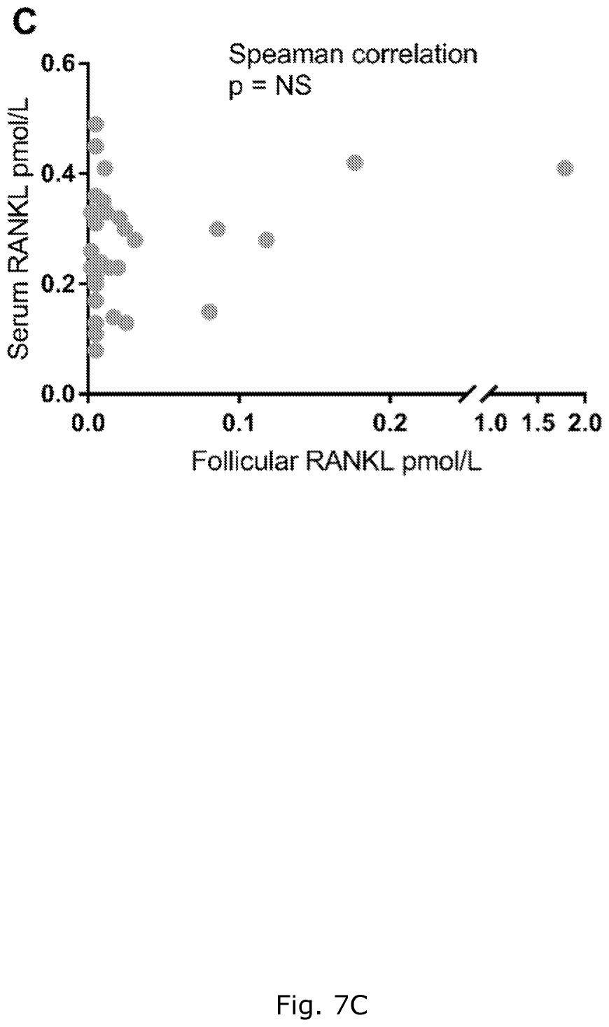

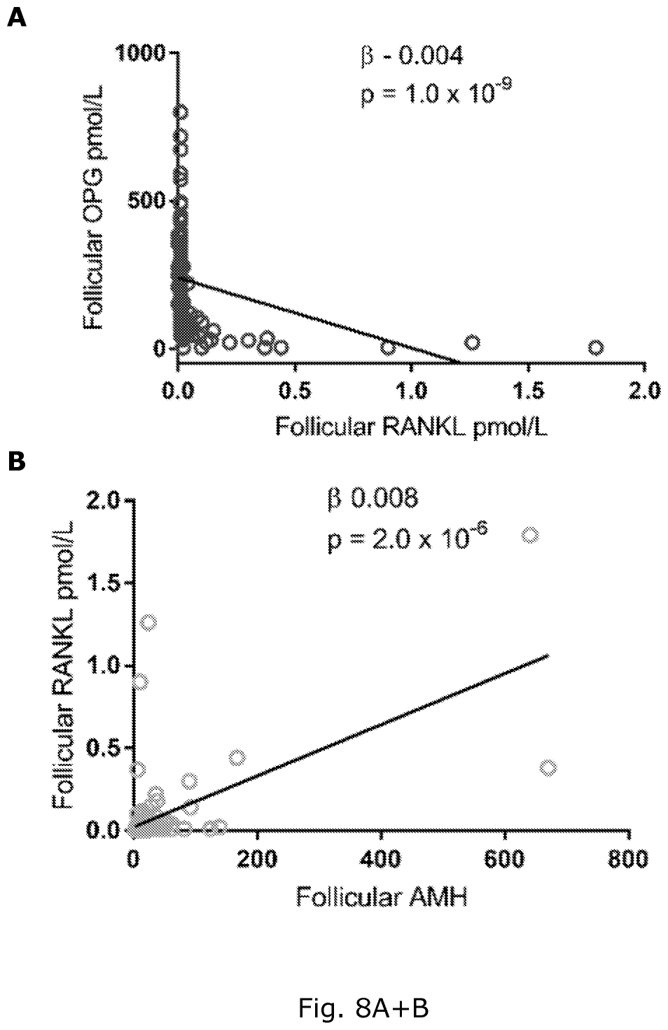

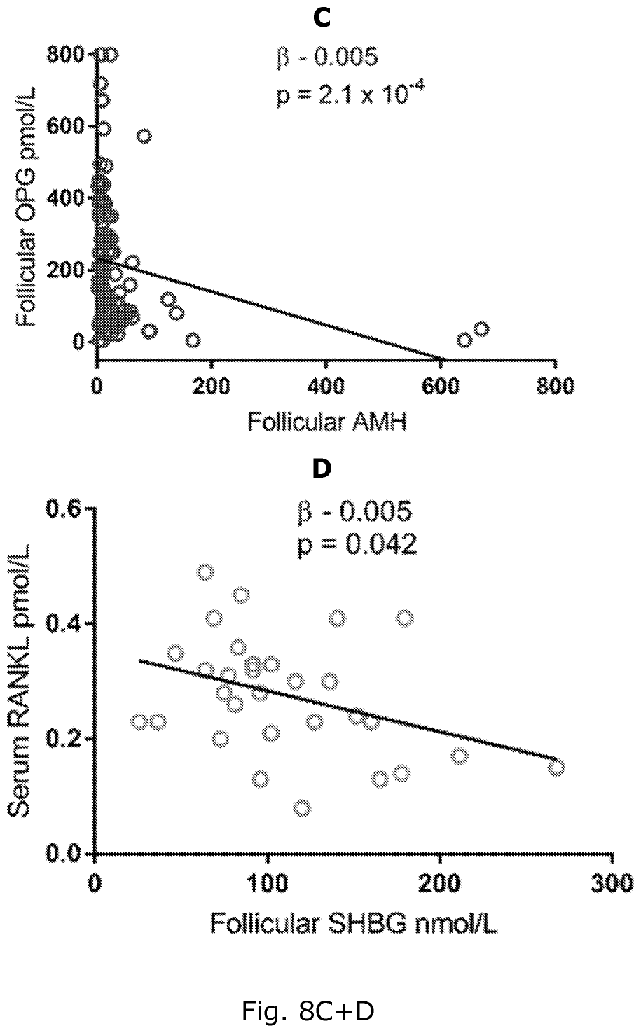

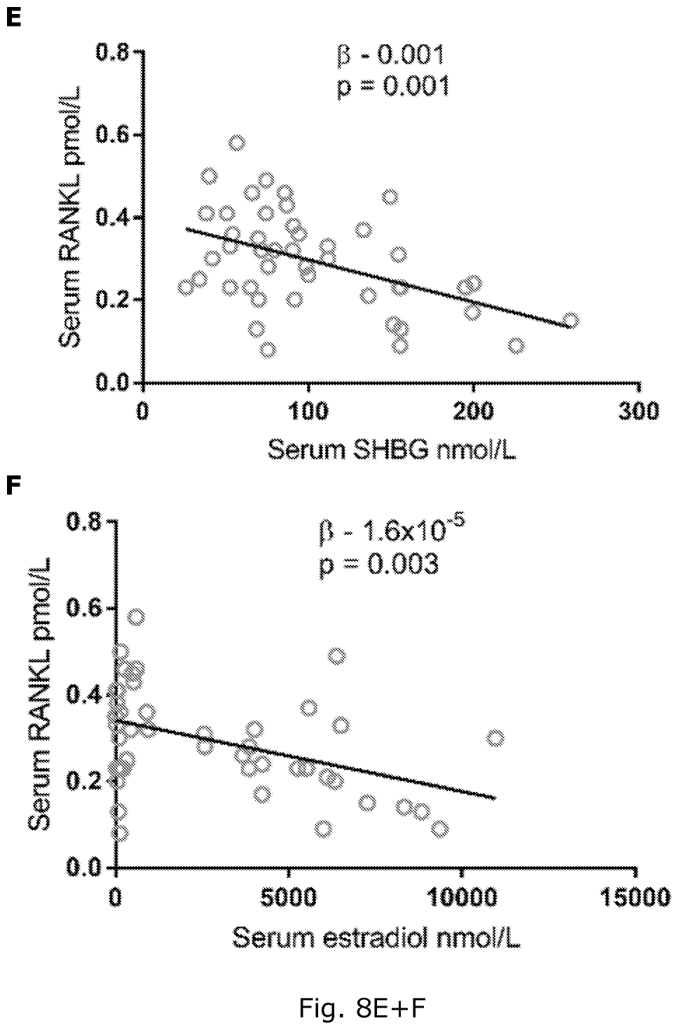

RANKL and OPG were measurable in follicular fluid and RANKL also in serum. 10 women had follicular RANKL below limit of detection, whereas all serum RANKL levels were above limit of detection. Follicular RANKL and OPG were both stable when measured repeatedly in the same woman in two different cycles of IVF/ICSI (FIGS. 7A and 7B). Follicular- and serum RANKL was not correlated (FIG. 7C). Follicular RANKL was inversely associated with follicular OPG (b −0.004, p=1.0×10⁻⁹, FIG. 8A) and significantly associated with AMH (b 0.008, p=2×10⁻⁶, FIG. 8B), but not with other hormones. OPG was inversely associated with AMH (b −0.005, 2.1×10⁻⁴, FIG. 8C), but with none of the other hormones. Serum RANKL was not associated with follicular RANKL nor follicular OPG. However, serum RANKL was inversely associated with follicular SHBG (b −0.005, p=0.042, FIG. 8D), inversely associated with serum SHBG (b −0.001, p=0.001, FIG. 8E) and inversely associated with serum estradiol (b −1.6×10⁻⁵, p=0.003, FIG. 8F).

RANKL, OPG and Clinical Outcomes

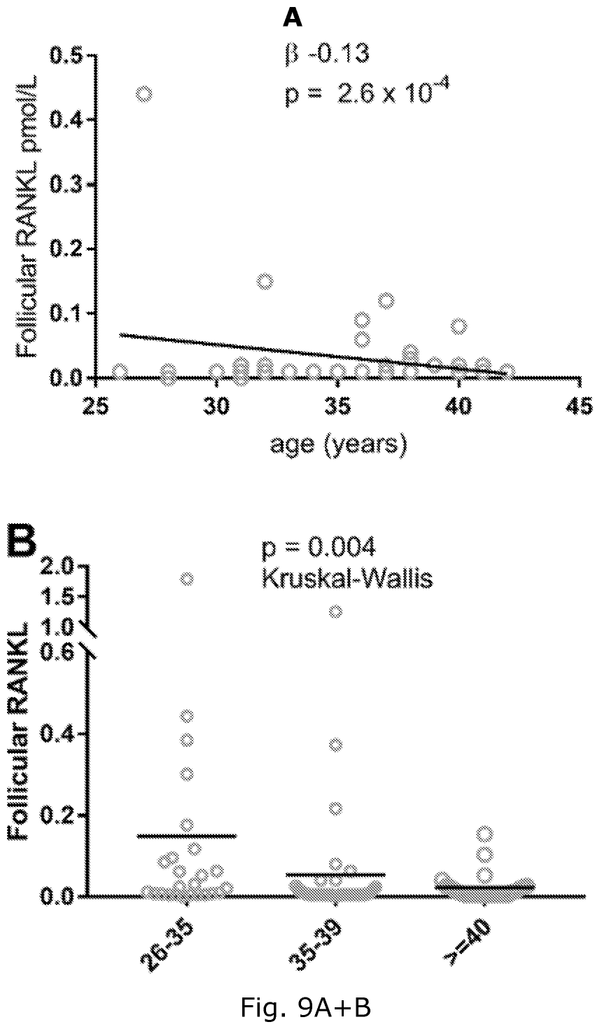

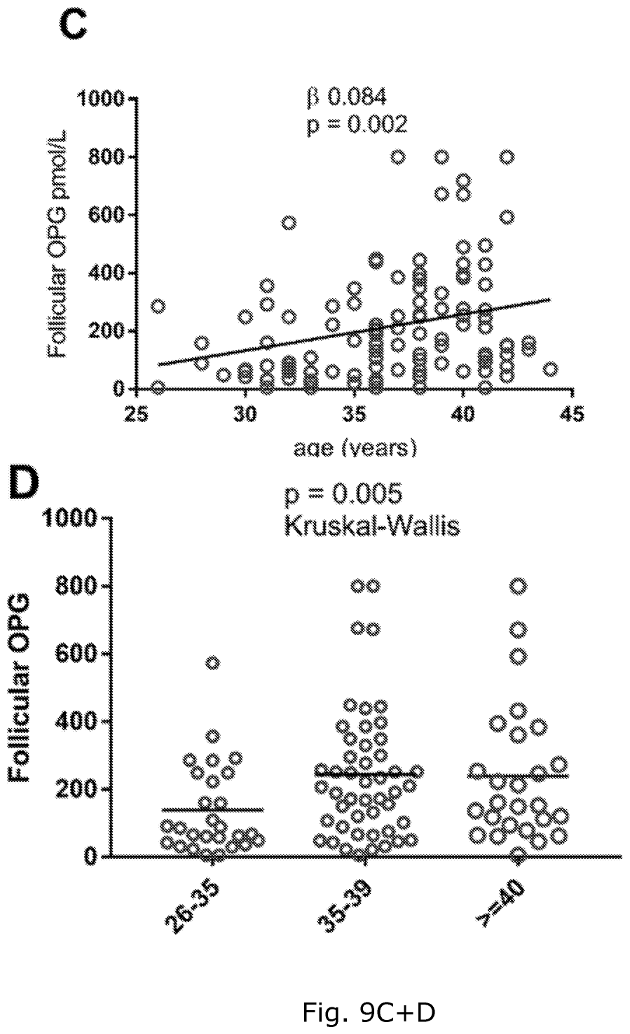

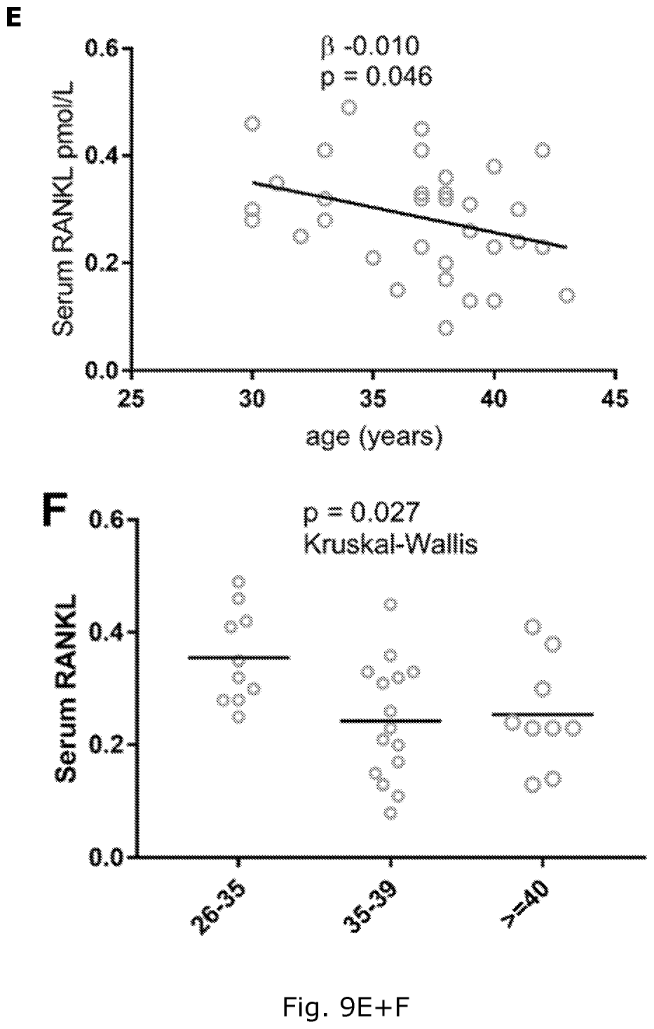

Follicular RANKL was further inversely associated with female age (b −0.13, p=2.6×10⁻⁴, FIG. 9A-B), where follicular OPG was positively associated with age (b 0.084, p=0.002, FIG. 9C-D). Neither RANKL nor OPG was associated with BMI, we did however only have information on BMI from 116 of the women.

As with follicular RANKL, serum RANKL was also inversely associated with age (b −0.010, p=0.046, FIG. 9E-F).

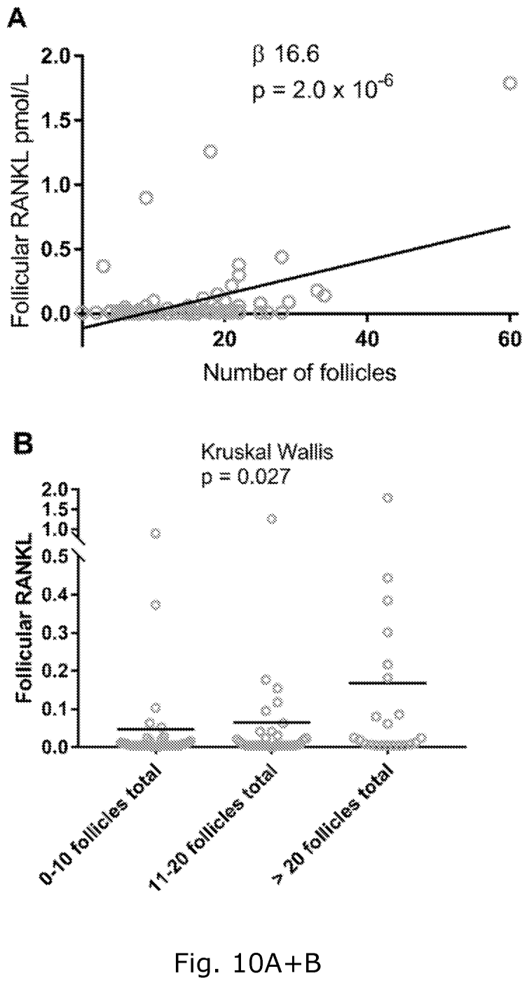

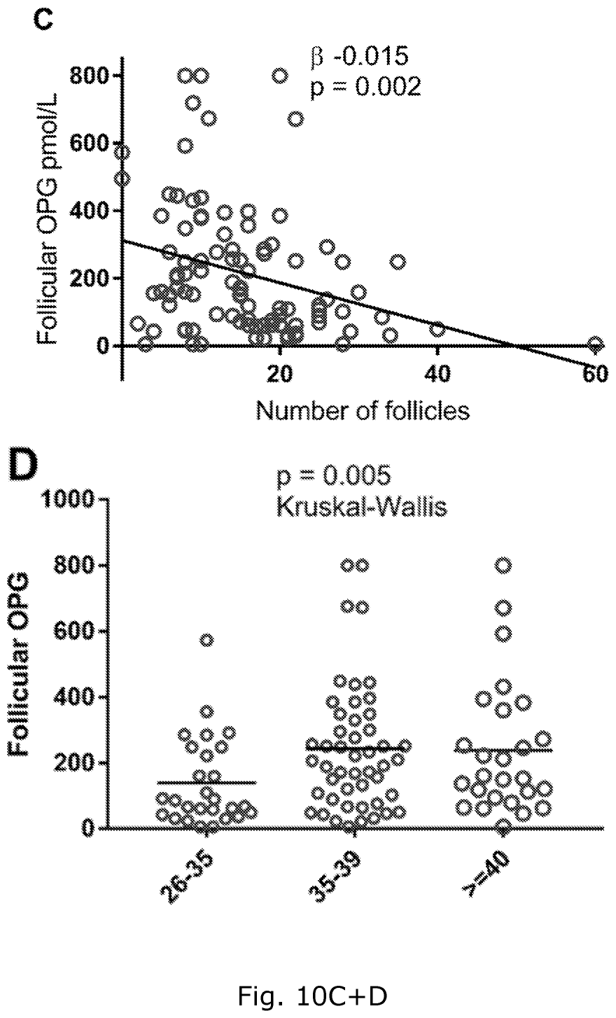

Number of follicles naturally matured by the women, before hormonal stimulation was significantly associated with follicular RANKL (b 16.6, p=2×10⁻⁶, FIG. 10A-B) and inversely associated with follicular OPG (b −0.015, p=0.002, FIG. 10C-D), but not with serum RANKL.

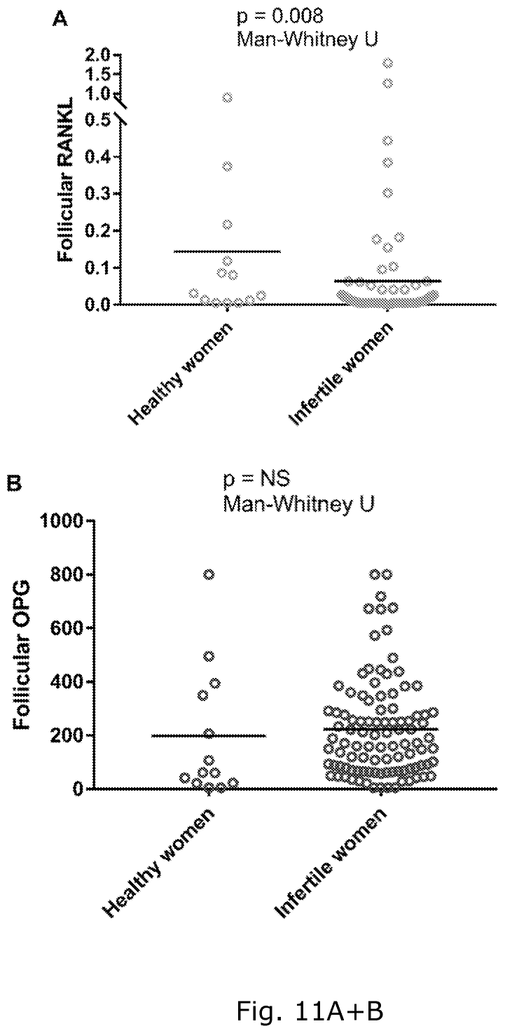

Comparing women having an infertility diagnosis with those who were gynecological healthy, but with an infertile spouse, showed that the healthy women had significantly higher follicular RANKL levels (p=0.008, Man-Whitney U), but comparable levels of follicular OPG and comparable remaining follicular hormones and serum hormones (FIG. 11A-B).

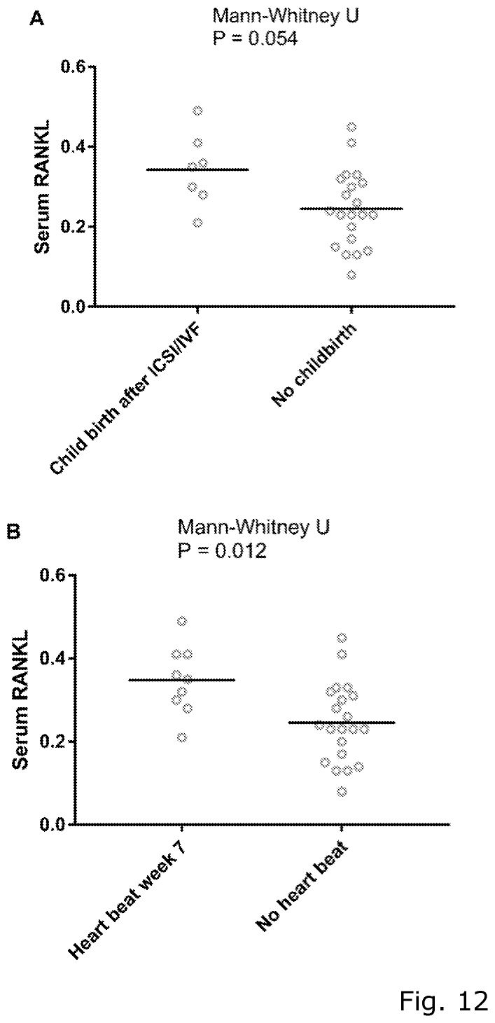

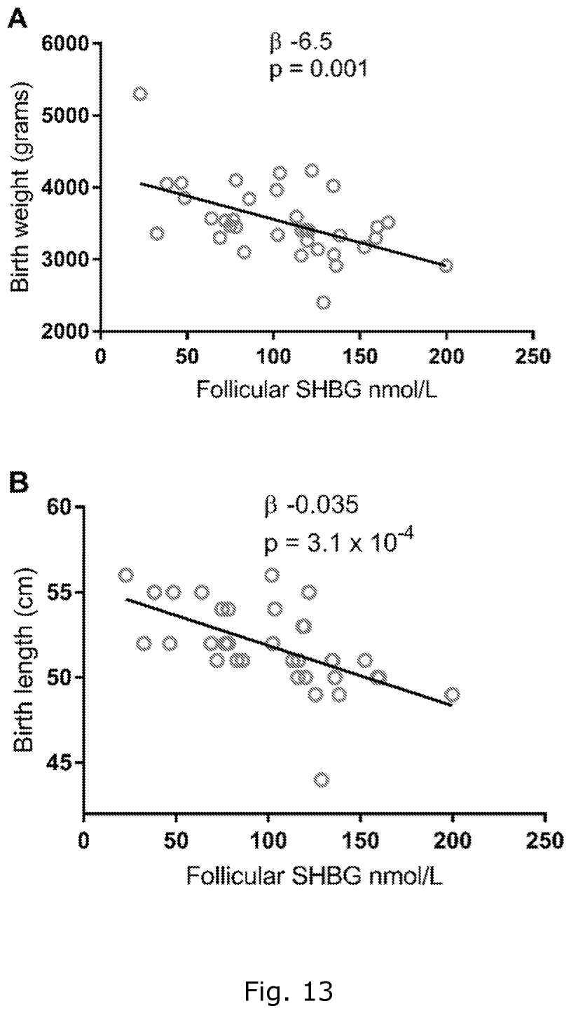

Clinical pregnancies obtained after ICSI and IVF resulting in live birth showed that woman who gave birth had borderline significantly higher serum RANKL, compared to those who became pregnant but had an early spontaneous abortion (p=0.054 Man-Whitney U, FIG. 12A). This shows that high RANKL is linked with successful pregnancy outcome and not just pregnancies. Furthermore, woman who became pregnant after IVF or ICSI and progressed to week seven with a positive fetal heartbeat, verified by ultrasonography, had significantly higher serum RANKL than those who became pregnant but didn't progress to week seven (p=0.012 Man-Whitney U, FIG. 12B). Follicular as well as serum levels of RANKL, OPG and all other hormones does not differ between these groups of women. Evaluating outcome of the children (birth weight, birth length and gestational age) in relation to follicular hormones revealed no relation to RANKL nor to OPG. We did, however, find a strong inverse association between follicular SHBG and birth weight (b −6.5, p=0.001, FIG. 13A) and birth length (b −0.035, p=3.1×10⁻⁴, FIG. 13B). Type of protocol used in ICSI/IVF did not alter serum-nor follicular levels of sRANKL or OPG.

Conclusion

RANKL is produced locally in the female reproductive tract and follicles and high RANKL is associated with increased fertility defined by higher number of follicles and serum AMH. Low follicular levels of RANKL is linked with reproductive problems and infertility, while very high RANKL is found in women with polycystic ovarian syndrome.

We were able to measure both RANKL and OPG in follicular fluid from women undergoing ART. Both RANKL and OPG showed to be stable over time, from one aspiration cycle to the next. Serum and follicular levels of RANKL were within the same range, however follicular RANKL was in general lower than serum levels, it reached a higher maximum and was also under limit of detection in a minority of the women. Serum levels of sRANKL were a little higher than in other cohorts (Sarink Et al. Cancer Epidemiol Biomarkers Prev. 2019 Oct.; 28(10):1746-1754), which can be explained by the younger age in this current cohort. OPG levels in follicular fluid was approximately 25 times higher than serum OPG levels in women).

Despite serum- and follicular levels of sRANKL being within the same range, we saw no correlation between the two, which implicates that the level of sRANKL in follicular fluid is locally and strictly regulated and not just a mirror of serum levels. We saw an inverse relationship between age of the woman and both her serum- and follicular RANKL level. The increasing follicular OPG levels with increasing age, mirror the finding in serum OPG in women, reported by others (Sarink Et al. Cancer Epidemiol Biomarkers Prev. 2019 Oct.; 28(10):1746-1754). We did not find a link between serum- and/or follicular levels of sRANKL with BMI. We found an interesting link between number of follicles naturally matured by the woman and her follicular RANKL level. We further showed a linear relationship between follicular sRANKL and follicular AMH. AMH is produced by the granulosa cells in the pre antral- and small antral follicles in the ovary and thus reflects number of follicles under maturation.

In addition, we showed that women who were reproductively healthy had higher follicular sRANKL levels than those who were infertile, but comparable levels of all other follicular- and serum hormones, including AMH. This might indicate that follicular sRANKL levels is a more sensitive marker of reproductive health. Women who became pregnant during IVF/ICSI did actually have a better chance of carrying the child to visual heart beat in week 7 and further on to giving birth, without spontaneous abortion, if she had higher levels of systemic serum RANKL at the time of conception. As discussed in example 1, impaired expression of RANKL in mice lead to dysfunction in stromal cells and to fetal loss. Thus, it is likely that low RANKL activity is associated with increased risk of abortions. This suggests that stimulation of RANKL production before and during ART may improve the chance for spontaneous pregnancy.

Local versus systemic RANKL levels seem to play a role at different timepoints in the reproductive circle of the woman.

### Example 4—In Vivo Mice Study—PTH

Aim of Study

To determine in vivo in mice the effect of a RANKL inducer, exemplified by PTH, on female fertility according to the present invention.

Materials and Methods

Study Design:

Twelve (12) female C57BL/6 mice will be allocated to two treatment groups (n=6, n=6) and administered with test article according to table 1. On study day 7, all females of each treatment group+3 male C57BL/6 mice will be allocated to the fertility study. In the fertility study pairs of 2 females will be housed with 1 male for a period of 5 days. During the 5 days all females are observed daily for vaginal plugs at least once per day. Administration of test articles will continue according to table 1 during the fertility study (days 7 to 11). Presence of vaginal plug, apparent pregnancy, and litter size will be recorded for each female. Females and litter will be terminated at the end of the fertility study and the weight of the pups will be determined.

Administration of Dose Solution:

Administration of test article i.p. (group 1) or vehicle i.p. (group 2) will be conducted 2 times a week for a period of 12 days.
Gr.1: 6 mice, i.p. dosing 2 times weekly for 12 days
Gr.2: 6 mice, i.p. dosing 2 times weekly for 12 days

TABLE 1
Administration

| Gr. No. | Mice (n) | Test article 1 (ID) | Dose | Route, dose/week |
|---|---|---|---|---|
| 1 | 6 | PTH | 5 μg/kg | i.p., 5 |
| 2 | 66 | Vehicle | n.a. | i.p., 2 |

Blood Sampling:

One blood sample will be harvested on study day 12 from all female mice in the study. The blood samples will be drawn by sublingual vein puncture.

Termination:
female mice from the fertility study, will be terminated on nominal study day 12. Twelwe (12) mice will be euthanized after giving birth to litters

Results

Injection of OPG in Healthy Mice:

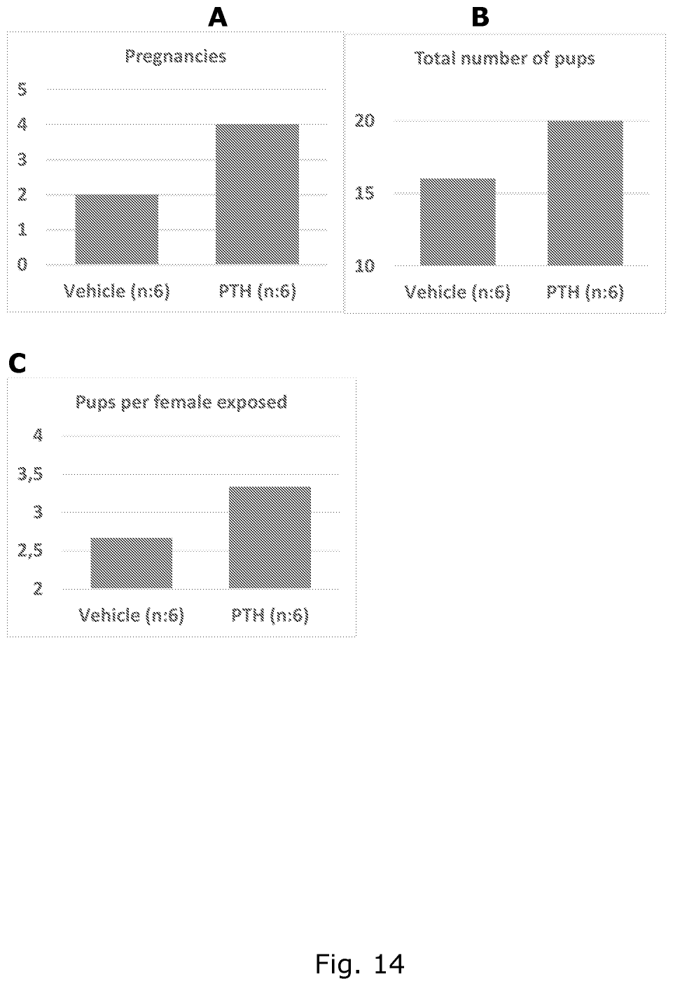

Short-term PTH treatment had a small but significant effect on ovary weight suggesting an effect on ovarian function p<0.05) (data not shown). Moreover, PTH increased pregnancy rate (FIG. 14A), number of pups (FIG. 14B) and number of pups per female in the PTH group compared with vehicle treated mice (FIG. 14C).

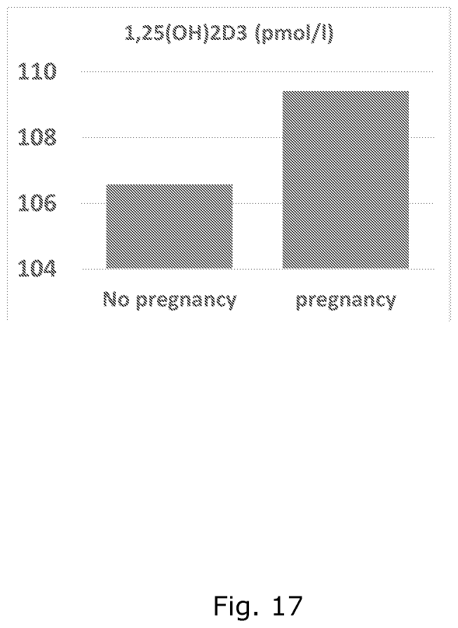

Further, Follicular fluid 1,25D3 levels (another RANKL stimulator) in follicular fluid is higher in women obtaining pregnancies validated by ultrasound after IVF compared with women having no pregnancy (FIG. 17).

Conclusion

Short manipulation of the RANKL signaling pathway by injecting of the well-characterized RANKL inducer PTH had a beneficial effect on fertility in normal mice. PTH treatment doubled pregnancy rates and increased number of pups and number of pups born per female exposed compared with vehicle, which demonstrates that induction of RANKL by PTH prior to and during reproduction is beneficial for live birth rate. The effect could in theory be exerted in many tissues as PTH treatment is not confined for reproductive tissues. However, the significant change in ovary weight indicates that part of the effect is mediated directly in the ovary.

The above data is supported by the study showing that Follicular fluid 1,25D3 levels (another RANKL stimulator) in follicular fluid is higher in women obtaining pregnancies validated by ultrasound after IVF compared with women having no pregnancy.

### Example 5—In Vivo Mice Study—OPG

Aim of Study

To determine in vivo in mice the effect of a systemic RANKL inhibitor exemplified by OPG on female fertility according to the present invention.

Materials and Methods

Study Design:

Twenty eight (28) female C57BL/6 mice will be allocated to 2 treatment groups (n=12, n=16) and administered with test article according to table 1. On study day 7, 6 females of each treatment group+6 male C57BL/6 mice will be allocated to the fertility study In the fertility study pairs of 2 females will be housed with 1 male for a period of 5 days. During the 5 days all females are observed daily for vaginal plugs at least once per day. Administration of test articles will continue according to table 1 during the fertility study (days 7 to 11). Presence of vaginal plug, apparent pregnancy, and litter size will be recorded for each female. Females and litter will be terminated at the end of the fertility study and the weight of the pups will be determined. Remaining female mice (n=6, and 10) not enrolled in the fertility study will be dosed as indicated in Table 2 and terminated on day 12. At terminations on day 12 tissues from all non-mated females will be isolated and weighed (ovaries (×2), salpinges (×2), uterus, kidneys (×2), tibial bones (×2)). Besides tissue samples, serum samples will also be provided from all female mice in the study (also day 12).

Administration of Dose Solution:

Administration of test article i.p. (group 1) or vehicle i.p. (group 2) will be conducted 2 times a week for a period of 12 days.
Gr.1:
12 mice, i.p. dosing 2 times weekly for 7 days
   1a: 6 mice, i.p. dosing 2 times weekly for further 5 days
   1b: 6 mice, i.p. dosing 2 times weekly for further 5 days, fertility arm
Gr.2:
16 mice, i.p. dosing 2 times weekly for 7 days
   2a: 10 mice, i.p. dosing 2 times weekly for further 5 days
   2b: 6 mice, i.p. dosing 2 times weekly for further 5 days, fertility arm

TABLE 2
Administration

| Gr. No. | Mice (n) | Test article 1 (ID) | Dose | Route, dose/week |
|---|---|---|---|---|
| 1 | 12 | OPG (cyt-177-c) | 1 mg/kg | i.p., 2 |
| 2 | 16 | Vehicle | n.a. | i.p., 2 |

Blood Sampling:

One blood sample will be harvested on study day 12 from all female mice in the study. The blood samples will be drawn by sublingual vein puncture.

Tissue Sampling and Tissue Preservation:

Females not enrolled in the fertility study will be euthanized on day 12. The following tissues will be dissected free and weighed from each mouse: Ovaries (×2), salpinges (×2), uterus, kidneys (×2), tibial bones (×2). Applied for ovaries, salpinges, and kidneys: one from each mouse will be preserved by freezing and one will be preserved by formalin fixation.

Termination:

Female mice from the non-mated study arm and 12 male mice from the fertility study, will be terminated on nominal study day 12. Twelve (12) mice will be euthanized after giving birth to litters, and no later than study day 42. Pups will be weighed after birth and euthanized.

Results

Injection of OPG in Healthy Mice:

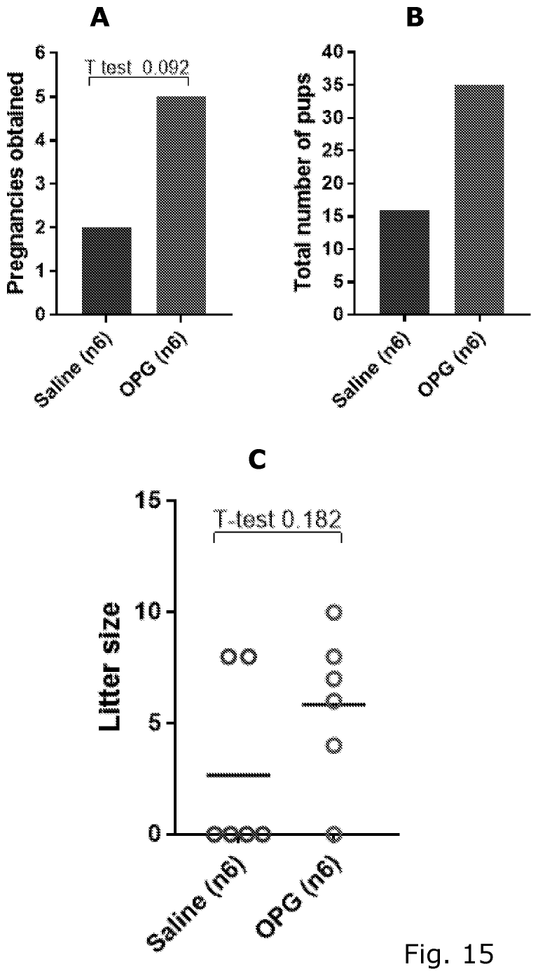

Short term OPG treatment had no effect on organ size (data not shown) but increased pregnancies obtained (FIG. 15A), total number of pubs (FIG. 15B) and litter size (FIG. 15C) with no ovarian problems, this effect may be mediated through the salpinges and the uterus where RANKL inhibition may results in increased implantation and better survival of the pups as indicated by the higher litter size. There was no statistically significant effect on serum AMH levels in OPG treated mice but a tendency indicating that RANKL is a powerful regulator of maturation of follicles.

Conclusion

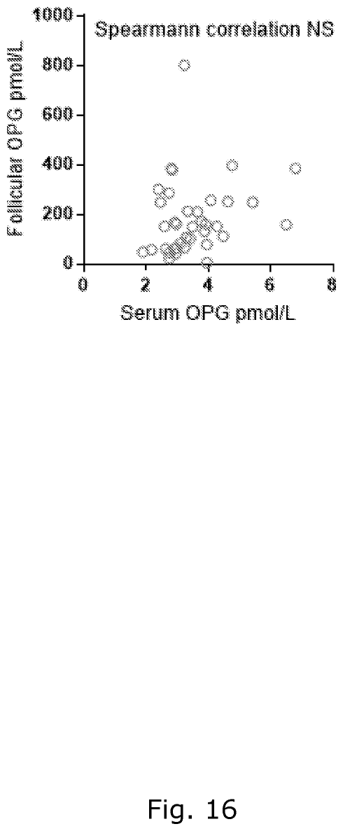

We know that OPG will not come into the follicular fluid and exert an effect there because OPG levels in follicular fluid is >100 fold higher than in serum and not influenced by OPG (FIG. 16). However, OPG will block RANKL in all extragonadal tissues including uterus.

Short manipulation of the RANKL pathway in all tissues accessibly by the RANKL inhibitor OPG has no effect on organ weight but increases pregnancies, number of pups and litter size and a tendency towards increased serum AMH, which supports that suppression of RANKL outside the follicle may improve female reproductive function and increase the likelihood of a live birth. The effect on live birth is most likely due to an effect on implantation and survival in the uterus.

1. A method for treating infertility in a female subject, or for improving the fertility potential in female subject, said method comprising administering a therapeutically effective amount of a compound selected from the group consisting of:
a stimulator of (ovarian) RANKL expression;
an agonist or inducer/activator of RANKL;
an antagonist or inhibitor of osteoprotegerin (OPG);
RANKL; and
an inhibitor of RANKL.

2-40. (canceled)

41. The method according to claim 1, wherein the compound is selected from the group consisting of:
a stimulator of RANKL expression;
an agonist or inducer/activator of RANKL;
an antagonist or inhibitor of osteoprotegerin (OPG); and
RANKL.

42. The method according to claim 1, wherein the female subject has
primary ovarian failure or primary ovarian insufficiency” (POI); and/or
old age just before menopause, and/or
low AMH levels.

43. The method according to claim 1, wherein said compound is a stimulator of ovarian RANKL expression.

44. The method according to claim 1, wherein the compound is selected from the group consisting of PTH, PTHRP, vitamin d, 25OHD, 1.25OH2D3, 24,25OH2D3, Etalpha, IL-1, TNF, FSH, LH, a SIK inhibitor, YKL-05-099 and combinations thereof.

45. The method according to claim 1, wherein the compound is selected from the group consisting of IL-1, IL-6, IL-11, IL-17, TNF-α, vitamin D, Ca²⁺, parathyroid, glucocorticoids, prostaglandin E2, and immunosuppressive drugs.

46. The method according to claim 1, wherein the compound is LH/hCG or FSH.

47. The method according to claim 1, wherein the compound is PTH.

48. The method according to claim 1, wherein the compound is selected from the group consisting of:
a stimulator of RANKL expression;
an agonist or inducer/activator of RANKL;
an antagonist or inhibitor of osteoprotegerin (OPG); and
RANKL;
wherein the female subject has
primary ovarian failure or primary ovarian insufficiency” (POI); and/or
old age just before menopause, and/or
low AMH levels.

49. The method according to claim 1, wherein the compound is an inhibitor of RANKL.

50. The method according to claim 1, wherein the female subject has Polycystic ovarian syndrome (PCOS).

51. The method according to claim 1, wherein the female subject has suffered multiple abortions and/or missed pregnancies.

52. The method according to claim 1, wherein the female subject has endometriosis.

53. The method according to claim 1, wherein the inhibitor of RANKL inhibits the binding of RANK to RANKL.

54. The method according to claim 1, wherein the inhibitor of RANKL is an antibody or antigen binding domain, which regulate the interaction between RANKL and RANK.

55. The method according to claim 1, wherein the compound is selected from the group consisting of Denosuomab, Cyt-177-c (OPG-FC) and OPG.

56. The method according to claim 1, wherein the compound is selected from the group consisting AS2676293, ABD328, ABD345, SPD-304, and E09241 and active fragments and analogues thereof.

57. The method according to claim 1, wherein the compound is an inhibitor of RANKL;
wherein the female subject has:
polycystic ovarian syndrome (PCOS);
suffered multiple abortions and/or missed pregnancies; and/or
endometriosis.

58. A method for predicting the fertility potential of a female subject, the method comprising:
determining the level of RANKL and/or OPG in a sample of follicular fluid or blood from the female subject;
comparing said level(s) to one or more corresponding reference levels; and
predicting that said subject is unlikely to have normal fertility potential when:
said RANKL level is below said corresponding reference level; and/or
said OPG is above said corresponding reference level(s);
or predicting that said subject is likely to have a normal fertility potential when:
said RANKL level is equal or above said corresponding reference level; and/or
said OPG level is equal to or below said corresponding reference level,
wherein if the fertility potential of the female subject is determined unlikely to be normal, initiating a treatment against the female infertility.

59. The method according to claim 58, wherein said treatment comprises administration of a compound selected from the group consisting of:
a stimulator of RANKL expression; and/or
an agonist or inducer/activator of RANKL; and/or
an antagonist or inhibitor of osteoprotegerin (OPG); and/or
RANKL; and
an inhibitor of RANKL.


SEQUENCE LISTING (8 sequences)

SEQ ID NO: 1 - DNA, 20 bp, Artificial Sequence
  Primer
ctgggagcgcaggttaaata

SEQ ID NO: 2 - DNA, 26 bp, Artificial Sequence
  Primer
gccaataattaaaatactgcaggaaa

SEQ ID NO: 3 - DNA, 20 bp, Artificial Sequence
  Primer
ctcagcttccagaggactgc

SEQ ID NO: 4 - DNA, 20 bp, Artificial Sequence
  Primer
gtgggctctatggcttctga

SEQ ID NO: 5 - DNA, 24 bp, Artificial Sequence
  Primer
cacgtgcagccgtttaagccgcgt

SEQ ID NO: 6 - DNA, 25 bp, Artificial Sequence
  Primer
ttcccattctaaacaacaccctgaa

SEQ ID NO: 7 - DNA, 21 bp, Artificial Sequence
  Primer
cctggaaaatgcttctgtccg

SEQ ID NO: 8 - DNA, 21 bp, Artificial Sequence
  Primer
cagggtgttataagcaatccc